(19) United States
(12) Patent Application Publication
(10) Pub. No.: US 2015/0031565 A1
(43) Pub. Date: Jan. 29, 2015

(54) DETERMINATION OF THE IDENTITIES OF SINGLE NUCLEOTIDE POLYMORPHISMS, POINT MUTATIONS AND CHARACTERISTIC NUCLEOTIDES IN DNA

(71) Applicants: Hong Xue, Tseung Kwan O (HK); Jeffrey Tze-Fei Wong, Tsuen Wan (HK)

(72) Inventors: Hong Xue, Tseung Kwan O (HK); Jeffrey Tze-Fei Wong, Tsuen Wan (HK)

(21) Appl. No.: 14/337,450

(22) Filed: Jul. 22, 2014

Related U.S. Application Data
(60) Provisional application No. 61/858,342, filed on Jul. 25, 2013.

(51) Int. Cl. C12Q 1/68

(52) U.S. Cl. CPC C12Q 1/6858
USPC 506/9; 435/6.11

(57) ABSTRACT

A method of genotyping single nucleotide polymorphisms (“SNP”) and point mutations in nucleic acid based on chain extension by polymerase. This invention is based on the fact that the neighboring sequence immediately 3′ adjacent to the site is known, and the nucleoside immediately 5′ adjacent to any SNP/point mutation site is also known. An extension primer complementary to the sequence directly adjacent to the SNP on the 3′ side of a target polynucleotide is used for chain extension. Up to four different polymerase reaction mixtures are provided in separate reaction containers, each containing one different potentially chain-extending Bridging Nucleotide. A Reporting Nucleotide having a base complementary to the nucleotide directly adjacent to the SNP on the 5′ side of the target polynucleotide may also be added to each reaction container.

### RELATED APPLICATIONS

This application claims the benefit of U.S. Provisional Patent Application Ser. No. 61/858,342, entitled DETERMINATION OF THE IDENTITIES OF SINGLE NUCLEOTIDE POLYMORPHISMS, POINT MUTATIONS AND CHARACTERISTIC NUCLEOTIDES IN DNA, filed on Jul. 25, 2013, the entire content of which is hereby incorporated by reference.

### BACKGROUND

The present invention relates to a method of determining or genotyping single nucleotide polymorphisms (“SNP”), point mutations and characteristic nucleotides in a nucleic acid molecule template. This method utilizes a null, double or longer base extension of an oligonucleotide primer to identify an SNP. The oligonucleotide primer is complementary to the DNA molecule template that contains the SNP or point mutation. The predicted extension length of the oligonucleotide extension primer is compared with the experimentally measured extension length, or the predicted amount of chemical label incorporation into the oligonucleotide extension primer is compared with the experimentally measured amount of incorporation, in order to determine the target nucleotide which can be an SNP, a point mutation or a characteristic nucleotide.

The human genome has been sequenced and the future efforts in genetics will compare the sequences of different individuals in order to understand human diseases. It is believed that there is about one polymorphism per 1,000 bases, which makes single nucleotide polymorphisms (“SNP”) and point mutations the most abundant type of genetic variations. The high density of SNP and point mutations in genomes make them powerful tools for mapping and diagnosing disease-related alleles. Although it appears that most SNP's and point mutations occur in non-coding regions, many SNP's and point mutations occur in exons and introns. The SNP's and point mutations have a number of properties of interest. Since SNP's and point mutations are inherited, such aberrant sequences can be used to determine genetic defects, such as deletions, insertions and mutations that may involve one or more bases in selected genes, or the genetic basis of inherited traits. Rather than isolating and sequencing the target gene, it is sufficient to identify only the SNP involved. Additionally, SNP's and point mutations can be used in forensic medicine to positively identify individuals. While other genetic markers are available, the large number of SNP's and point mutations and their extensive distribution in the chromosomes make the SNP's attractive targets. Also, by determining a plurality of SNP's associated with a specific phenotype, one may use the SNP pattern as an indication of the phenotype, rather than requiring a determination of the genes associated with the phenotype.

Besides SNP's and point mutations in the human genome, deletions within a human DNA segment can be identified as reduced copies of characteristic nucleotides in the segment, and insertions can be identified as increased copies. Moreover, the identification of target nucleotides including SNP's, point mutations and characteristic nucleotides serves a useful purpose with non-human genomes, including the genomes of animals and plants, where target nucleotides provide a basis for distinguishing between different genetic species or varieties, and microorganisms and viruses, where target nucleotides provide a basis for confirming the presence of a particular bacterial or viral strain in a specimen, identifying drug-resistance genetic sites in bacteria and viruses, and assessing the proportions between different bacterial or viral strains in a specimen containing a mixed population of bacterial or viral strains.

The need to identify a large number of bases distributed over potentially centimorgans of DNA offers a major challenge. Any method should be accurate, reasonably economical in limiting the amount of reagents required and providing for a single assay that allows for differentiation of the different SNP or other target nucleotides. Many methods have been described for the detection of such genetic polymorphisms. For example, U.S. Pat. No. 6,110,709 describes a method for detecting the presence or absence of an SNP in a nucleic acid molecule by first amplifying the nucleic acid of interest, followed by restriction analysis and immobilizing the amplified product to a binding element on a solid support. Patent Publication WO9302212 describes another method for amplification and sequencing of nucleic acid in which dideoxy nucleotides are used to create amplified products of varying lengths. The varying length products are then separated and visualized by gel electrophoresis. Patent Publication WO0020853 further describes a method of detecting single base changes using tightly controlled gel electrophoretic conditions to scan for conformational changes in the nucleic acid caused by sequence changes. U.S. Pat. No. 6,972,174, (“the '174 patent”) the entire content of which is incorporated herein by reference, describes a method for identifying a nucleotide at a SNP site by first amplifying the nucleic acid of interest, and using DNA polymerase to elongate an extension primer that is complementary to the sequence on the 3′ side of a SNP site in a template DNA. The polymerase mixture contains one chain-terminating nucleotide having a base complementary to the nucleotide directly adjacent to the SNP on the 5′ side in the template DNA. The resultant elongation/termination reaction products are analyzed for the length of chain extension of the primer, or for the amount of label incorporation from a labeled form of the terminating nucleotide.

In order to screen a large number of different samples, there is a need for a method with high reliability as well as wide applicability. In particular, the use of chain-terminating nucleotides as described in for example the '174 patent precludes the use of many DNA polymerases which function either poorly or not at all toward the incorporation of chain-terminating dideoxyribonucleoside triphosphate (“ddNTP”) analogues into growing DNA oligonucleotide chains. Here, incorporation of the ddNTP Reporting Nucleotide immediately causes chain termination. Thus, the problem to be solved in the '174 patent is to provide a method of detecting SNP's or point mutations using a chain-terminating nucleotide, such as ddNTP. The '174 patent discloses the incorporation and detection of a label; however, the label is on a chain-terminating ddNTP. A dye-ddNTP substrate as employed in the '174 patent, where the dye brings fluorescence and the dideoxyribose brings about chain termination, in effect differs from dNTP the natural substrate of DNA polymerase by two counts, viz, the presence of the dye moiety and the substitution of dideoxyribose for deoxyribose. Interference due to the dye moiety can be minimized through the choice of the dye, but DNA polymerases in general do not really work well with ddNTP chain-terminators. Even DNA polymerases that catalyze the incorporation of terminating nucleotides into DNA primer chains commonly function with significantly unequal reaction rates toward different chain-terminating nucleotides. Therefore, the reliability and applicability of target nucleotide identification using methods that employ and rely on chain-terminating nucleotides are severely limited. The present invention provides a novel primer elongation or extension method for scoring single nucleotide polymorphisms and variations that does not require the use of any chain-terminating nucleotides at all.

### SUMMARY

The present invention relates to a method of identifying a single nucleotide polymorphism (“SNP”) or other target nucleotide in a nucleic acid molecule. This method utilizes base extensions of oligonucleotidyl extension primers that are complementary to the DNA template molecule containing the SNP (or more generally target nucleotide). The predicted extension length of the extension primer in the presence of a “Bridging Nucleotide” in the form of a dNTP or dNTP-R (where “dNTP” represents deoxynucleoside triphosphate, and R represents a reporting chemical label) that is complementary to the SNP nucleotide and therefore enables primer elongation across the target nucleotide, and a “Reporting Nucleotide” in the form of a dNTP-R that is complementary to the nucleotide directly adjacent to the SNP on the 5′ side in the template DNA, is compared with the experimental extension length to identify the target nucleotide. Moreover, the predicted amount of labeled deoxyribonucleoside monophosphate (“dNMP-R”) incorporated from the dNTP-R nucleotide(s) on to the extension primer can be compared with the amount of experimental incorporation to identify the SNP or target nucleotide.

In one embodiment of the present invention an oligonucleotidyl extension primer is hybridized to a DNA template such that the oligonucleotidyl extension primer is complementary to the sequence on the DNA template that is immediately adjacent to the known SNP or other target nucleotide on the 3′ side. The extension primer and the DNA template are hybridized together and form a hybridized-DNA. The extension primer can then be elongated under polymerization conditions that will yield a zero-base, two-base or longer elongation of the extension primer with the complementary nucleic acid bases on the DNA template that contains the target nucleotide acting as template for the extension. For example, adding a Bridging Nucleotide in the form of dNTP to a polymerization reaction mixture will bring about either zero extension in the event that the SNP on the DNA template is non-complementary to the Bridging Nucleotide, or an extension across the SNP nucleotide site in the event that the SNP is complementary to the Bridging Nucleotide. In the latter instance, by adding to the reaction mixture a second Reporting Nucleotide in the form of dNTP-R, in which the dNTP moiety is complementary to the nucleotide immediately 5′ adjacent to the SNP on the DNA template, the primer will be extended further across the adjacent nucleotide 5′ to the SNP site as well causing the incorporation of the nucleoside monophosphate dNMP-R moiety into the primer, and thereby rendering detectable on the extension primer the incorporated “R” reporter group. In the present invention, the same Reporting dNTP-R that is complementary to the 5′ adjacent nucleotide is used in polymerization reaction mixtures containing different Bridging Nucleotides, and the successful incorporation of a dNMP-R residue into the extension primer in a particular polymerization reaction mixture will indicate that the Bridging dNTP employed in that mixture is in fact complementary to the SNP nucleotide, thus revealing the identity of the SNP nucleotide on the DNA template. The successful incorporation of the dNMP-R is detected by means of the extended length of the extension primer, or the presence of 21 the dNMP-R on the elongated extension primer, or both. Accordingly the identity of a SNP or other target nucleotide within a haploid DNA template, a diploid DNA template that is homozygous with respect to the target nucleotide, or a diploid DNA template that is heterozygous with respect to the target nucleotide, can then be determined by utilizing a table of predicted lengths of the elongated extension primers in each of the reaction tubes, or a table showing the amounts of predicted incorporation of dNMP-R in each of the reaction tubes, or both. In the Reporting dNTP-R molecule employed, the “R” group can be any measurable group, e.g. any fluorescent dye, non-fluorescent dye or isotopic label covalently attached to the dNTP moiety, that does not interfere with the complementary pairing of the dNTP moiety and its participation in DNA polymerization. The “R” group can even be simply a hydrogen atom in the event that mass spectrometry is used to detect the addition of the dNMP-R residue on to the extension primer.

Another embodiment of the invention utilizes the method described above to detect a target nucleotide on a DNA template in a solid-phase mode. Such an application in solid phase would allow mass genetic screening to occur on a surface such as a DNA chip. For example, oligonucleotidyl extension primers of DNA, RNA, or peptide nucleic acid (“PNA”) with sequences complementary to a known sequence in a DNA template molecule on the 3′ side of an SNP or point mutation or characteristic residue can be coated on to a solid surface (e.g. glass, metal, plastic, nylon, beads or any other suitable matrices). The DNA template molecule can then be hybridized to the immobilized extension primer on the solid surface and serve as a template for elongation of the primer. As similarly described above, the addition of the appropriate Bridging dNTP's and Reporting dNTP-R's will extend the immobilized extension primers by zero, two or more bases. In the case of a chemically labeled Reporting dNTP-R, primer extension will lead to incorporation of dNMP-R to yield a detectably labeled primer. By employing experimental conditions under which the amount of incorporated dNMP-R is proportional to the amount of template DNA containing a particular target nucleotide, the amount of DNA template containing the particular target nucleotide can be estimated. Lack of labeled extension primers indicates the absence of any elongation. On this basis the presence or absence of a known SNP or point mutation or characteristic residue within a haploid, homozygous diploid, or heterozygous diploid DNA template can be determined by utilizing a table of predicted amounts of label incorporation from a Reporting dNTP-R on to the oligonucleotide primers in reaction mixtures containing different Bridging dNTP's.

The table for predicting the identity of for example an SNP comprises column-headings, row-headings and predicted lengths for an oligonucleotidyl extension primer. Individual column headings on the table represent the different reaction conditions employed for extending an oligonucleotide primer to zero, two or more nucleotides longer than the added primer to form the elongated primer. Individual row-headings on the table represent nucleic acid sequences with potential permutations of the SNP (or target nucleotide), and the predicted lengths of elongated oligonucleotidyl primers are listed at the intersection point of different columns and rows. By comparing the predicted length of the extended oligonucleotidyl extension primer and the observed length of the extension primer in each reaction mixture, one can use the table to identify the SNP(s) in a haploid or diploid DNA template. Alternately, instead of the predicted lengths for an oligonucleotidyl extension primer, the table can show the predicted amounts of dNMP-R residues incorporated into the oliogonucleotidyl extension primer. In this case the column headings represent the reaction conditions employed to give rise to the incorporation of zero, one, two, or more dNMP-R residues into the oligonucleotidyl extension primer.

### BRIEF DESCRIPTION OF DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a schematic of an embodiment of the present invention; a template DNA strand having a 3′ portion and a 5′ portion is hybridized with a complementary oligonucleotidyl extension primer having a 3′ portion and a 5′ portion; an SNP (or target nucleotide) is directly adjacent to the 3′ hydroxy terminus of the extension primer. The SNP site nucleotide N can take the allelic form of A, T, C or G. In this figure and in FIG. 7, X and Y represent unspecified nucleotide residues, of which directly opposing X and Y on opposite strands of a double helix are complementary. These components are incubated with a DNA polymerase, which can bring about an extension of the extension primer in the presence of an added Bridging Nucleotide if and only if the added Bridging Nucleotide is a deoxynucleoside triphosphate bearing a base that is complementary to the base on the SNP nucleotide N.

FIG. 2 shows a table that can be utilized to determine the identity of an SNP nucleotide in a haploid target site by chain length analysis of the extension primer in the DNA polymerase reaction shown in FIG. 1, wherein the headings of columns 2-5 represent the four different polymerase reaction mixes employed for extending the extension primer. Each of the A Mix, T Mix, G Mix and C Mix contains a Bridging Nucleotide that enables the extension primer to extend across the SNP site, and a Reporting Nucleotide that is complementary to the nucleotide 5′ adjacent to the SNP; the nature of the Bridging Nucleotide together with that of the Reporting Nucleotide determines the status of the expected extension reaction. Because in this embodiment all the sequences flanking the haploid SNP contain a G residue 5′ adjacent to the SNP, the Reporting Nucleotide selected is dCTP-R, where the covalently-linked R-group can be simply a hydrogen atom, or a chemical label such as a fluorescent dye, non-fluorescent dye or isotopic group. The C-mix, in which dCTP-R doubly serves as Bridging Nucleotide and Reporting Nucleotide, is appropriate for extending the primer across a G residue at the SNP site. Each of rows 2-5 in the table shows a representation of a DNA template sequence with potential permutations of the SNP site nucleotide, along with the predicted length of the extended primer in each of the four different reaction mixes.

FIG. 3 shows a table in an embodiment of the present invention that can be utilized to determine the identity of an SNP in a diploid DNA template by chain length analysis of the extension primer in the reaction shown in FIG. 1, wherein the headings of columns 2-5 represent different polymerase reaction mixes employed for primer extension; rows 2-11 in column 6 represent a DNA sequence with potential permutations of the nature of the nucleotide at a homozygous or heterozygous SNP site; and the predicted length of extension of extension primer occurring in each of the four different reaction mixes is indicated in the box at the intersection of a column-heading and a row-heading.

FIG. 4 shows a table in an embodiment of the present invention that can be utilized to determine the identity of an SNP in a haploid DNP template based on the incorporation of a labeled Reporting Nucleotide (dCMP-R in this instance) residue, carrying for example a fluorescent dye, non-fluorescent dye or isotopic label, into the extension primer in the reaction shown in FIG. 1, without regard for or analysis of the actual length of the primer elongation. The headings of columns 2-5 indicate different polymerase reaction mixes employed for primer extension; the headings of rows 2-5 in column 6 show a DNA template sequence with potential permutations of the SNP nucleotide at the haploidal SNP site; and the predicted amount of label incorporation into the extension primer is indicated in the box at the intersection of a column-heading and a row-heading; wherein 0=no label incorporation, 1×=incorporation of one dCMP-R residue per haploid SNP site, 2×=incorporation of two dCMP-R residues per haploid SNP site.

FIG. 5 shows a table in an embodiment of the present invention that can be utilized to determine the identity of an SNP nucleotide(s) in a diploid SNP site based on the incorporation of a labeled Reporting Nucleotide (dCMP-R in this instance) residue into the extension primer in the reaction shown in FIG. 1, without regard to or analysis of the actual length of the primer elongation. The headings of columns 2-5 indicate different polymerase reaction mixes employed for primer extension; the headings of rows 2-11 in column 6 show a DNA template sequence with potential permutations of the SNP nucleotide(s) at a homozygous or heterozygous SNP site. The predicted amount of label incorporation into the extension primer is indicated in the box at the intersection of a column-heading and the row-heading; wherein 0=no dCMP-R incorporation, 1×=incorporation of one dCMP-R residue per diploid SNP site; 2×=incorporation of two dCMP-R residues per diploid SNP site; 3×=incorporation of three dCMP-R residues per diploid SNP site, and 4×=incorporation of four dCMP-R residues per diploid SNP site.

FIG. 6 shows a schematic drawing in an embodiment of the present invention; a DNA molecule having a 3′ portion and a 5′ portion is PCR-amplified using oligonucleotidyl Primer 1 and Primer 2 to amplify a selected portion of a DNA template having an SNP (or target nucleotide) interposed between Primers 1 and 2.

FIG. 7 shows a continuation from FIG. 6, including a schematic drawing in an embodiment of the present invention; a DNA molecule having a 3′ portion and a 5′ portion is PCR-amplified using the oligonucleotidyl Primer 1 and Primer 2 shown in FIG. 6 to amplify a selected portion of a DNA template having an SNP (or target nucleotide) interposed between Primers 1 and 2; the amplified DNA template is hybridized with a complementary oligonucleotidyl Primer 3 having a 3′ and 5′ portion; an SNP (or target nucleotide) on the amplified DNA template is directly adjacent to the 3′ hydroxy terminus of Primer 3, which serves as extension primer in a polymerase-catalyzed extension across the SNP site in the presence of a dNTP Bridging Nucleotide that is complementary to the SNP site nucleotide. After crossing the SNP site, Primer 3 extension continues across the two A-residues 5′ to the SNP on the amplified DNA template in the presence, and only in the presence, of a complementary dTTP-R Reporting Nucleotide. Thereupon further extension calls for the presence of dGTP, complementary to the C residue 5′ to the two A-residues. Accordingly further extension across this C-residue is allowed if Bridging Nucleotide happens to be dGTP; otherwise it is disallowed, and the extension reaction comes to a stop after a 3-nucleotide long extension of Primer 3.

FIG. 8 shows a table in an embodiment of the present invention that can be utilized to determine the identity of an SNP in a haploid DNA template based on chain length analysis of Primer 3 in the polymerase reaction shown in FIG. 7, wherein the headings of columns 2-5 represent polymerase reaction mixes for primer extension; the headings of rows 2-5 in column 6 represent a DNA template sequence with potential permutations of the SNP nucleotide in the haploid DNA template; and the predicted length of extension of Primer 3 occurring in each of the four different reaction mixes is indicated in the box at the intersection of a column-heading and a row-heading.

FIG. 9 shows a table in an embodiment of the present invention that can be utilized to determine the identity of an SNP in a diploid DNA template by chain length analysis of Primer 3 in the polymerase reaction shown in FIG. 7, wherein the headings of columns 2-5 represent polymerase reaction mixes for primer extension; the headings of rows 2-11 in column 6 represent a DNA template sequence with potential permutations of the SNP nucleotide in the diploid DNA template; and the predicted length of extension of Primer 3 occurring in each of the four different reaction mixes is indicated in the box at the intersection of a column-heading and a row-heading.

FIG. 10 shows a table in an embodiment of the present invention that can be utilized to determine the identity of an SNP in a haploid DNA template based on incorporation of a labeled Reporting Nucleotide (dTMP-R in this instance) residue, carrying for example a fluorescent dye, non-fluorescent dye or isotopic label, into Primer 3 in the polymerase reaction shown in FIG. 7, without regard to or analysis of the actual length of the primer extension. The headings of columns 2-5 represent different polymerase reaction mixes employed for primer extension; the headings of rows 2-5 in column 6 represent a DNA template sequence with potential permutations of the SNP nucleotide. The predicted amount of label incorporation into Primer 3 occurring in each of the four reaction mixes is indicated in the box at the intersection of a column-heading and a row-heading; wherein 0=no label incorporation, 2×=incorporation of two dTMP-R residues per haploid SNP site, and 3×=incorporation of three dTMP-R residues per haploid SNP site.

FIG. 11 shows a table in an embodiment of the present invention that can be utilized to determine the identity of an SNP in a diploid DNA template based on incorporation of a labeled Reporting Nucleotide (dTMP-R in this instance) residue into Primer 3 in the polymerase reaction shown in FIG. 7, without regard to or analysis of the actual length of the primer extension; wherein the headings of columns 2-5 represent different polymerase reaction mixes employed for primer extension; the headings of rows 2-11 represent a DNA sequence with potential permutations of the SNP nucleotide in a homozygous or heterozygous diploid DNA template; and the predicted amount of label incorporation into Primer 3 occurring in each of the four reaction mixes is indicated in the box at the intersection of a column-heading and a row-heading; and 0=no label incorporation, 2×=incorporation of two dTMP-R residues per diploid SNP site; 3×=incorporation of three dTMP-R residues per diploid SNP site, 4×=incorporation of four dTMP-R residues per diploid SNP site, 5×=incorporation of five dTMP-R residues per diploid SNP site, and 6×=incorporation of six dTMP-R residues per diploid SNP site.

FIG. 12, similar to FIG. 8 except for the use of dNTP-R Bridging Nucleotides instead of dNTP Bridging Nucleotides, shows a table in an embodiment of the present invention that can be utilized to determine the identity of an SNP in a haploid DNA template based on chain length analysis of Primer 3 in the polymerase reaction shown in FIG. 7, wherein the headings of columns 2-5 represent polymerase reaction mixes for primer extension; the headings of rows 2-5 in column 6 represent a DNA template sequence with potential permutations of the SNP nucleotide in the haploid DNA template; and the predicted length of extension of Primer 3 occurring in each of the four different reaction mixes is indicated in the box at the intersection of a column-heading and a row-heading.

FIG. 13, similar to FIG. 9 except for the use of dNTP-R Bridging Nucleotides instead of dNTP Bridging Nucleotides, shows a table in an embodiment of the present invention that can be utilized to determine the identity of an SNP in a diploid DNA template by chain length analysis of Primer 3 in the polymerase reaction shown in FIG. 7, wherein the headings of columns 2-5 represent polymerase reaction mixes for primer extension; the headings of rows 2-11 in column 6 represent a DNA template sequence with potential permutations of the SNP nucleotide in the diploid DNA template; and the predicted length of extension of Primer 3 occurring in each of the four different reaction mixes is indicated in the box at the intersection of a column-heading and a row-heading.

FIG. 14, comparable to FIG. 10 except for the use of dNTP-R Bridging Nucleotides instead of dNTP Bridging Nucleotides, shows a table in an embodiment of the present invention that can be utilized to determine the identity of a SNP in a haploid DNA template based on incorporation of a labeled Reporting Nucleotide (dTMP-R in this instance) residue, carrying for example a fluorescent dye, non-fluorescent dye or isotopic label, into Primer 3 in the polymerase reaction shown in FIG. 7, without regard to or analysis of the actual length of the primer extension. The headings of columns 2-5 represent different polymerase reaction mixes employed for primer extension; the headings of rows 2-5 in column 6 represent a DNA template sequence with potential permutations of the SNP nucleotide. The predicted amount of label incorporation into Primer 3 occurring in each of the four reaction mixes is indicated in the box at the intersection of a column-heading and a row-heading; wherein 0=no label incorporation, 3×=incorporation of three dNMP-R residues per haploid SNP site, and 4×=incorporation of four dNMP-R residues per haploid SNP site.

FIG. 15, comparable to FIG. 11 except for the use of dNTP-R Bridging Nucleotides instead of dNTP Bridging Nucleotides, shows a table in an embodiment of the present invention that can be utilized to determine the identity of an SNP in a diploid DNA template based on incorporation of a labeled Reporting Nucleotide (dTMP-R in this instance) residue into Primer 3 in the polymerase reaction shown in FIG. 7, without regard to or analysis of the actual length of the primer extension; wherein the headings of columns 2-5 represent different polymerase reaction mixes employed for primer extension; the headings of rows 2-11 represent a DNA sequence with potential permutations of the SNP nucleotide in a homozygous or heterozygous diploid DNA template; and the predicted amount of label incorporation into Primer 3 occurring in each of the four reaction mixes is indicated in the box at the intersection of a column-heading and a row-heading; and 0=no label incorporation, 3×=incorporation of three dNMP-R residues per diploid SNP site; 4×=incorporation of four dNMP-R residues per diploid SNP site, 6×=incorporation of six dNMP-R residues per diploid SNP site, and 8×=incorporation of eight dNMP-R residues per diploid SNP site.

FIG. 16, comparable to FIG. 8 except for the use of dNTP-R Bridging Nucleotides instead of dNTP Bridging Nucleotides and omission of Reporting Nucleotide from the four mixes, shows a table in an embodiment of the present invention that can be utilized to determine the identity of an SNP in a haploid DNA template based on chain length analysis of Primer 3 in the polymerase reaction shown in FIG. 7, wherein the headings of columns 2-5 represent polymerase reaction mixes for primer extension; the headings of rows 2-5 in column 6 represent a DNA template sequence with potential permutations of the SNP nucleotide in the haploid DNA template; and the predicted length of extension of Primer 3 occurring in each of the four different reaction mixes is indicated in the box at the intersection of a column-heading and a row-heading.

FIG. 17, similar to FIG. 9 except for the use of dNTP-R Bridging Nucleotides instead of dNTP Bridging Nucleotides and omission of Reporting Nucleotide from the four mixes, shows a table in an embodiment of the present invention that can be utilized to determine the identity of an SNP in a diploid DNA template by chain length analysis of Primer 3 in the polymerase reaction shown in FIG. 7, wherein the headings of columns 2-5 represent polymerase reaction mixes for primer extension; the headings of rows 2-11 in column 6 represent a DNA template sequence with potential permutations of the SNP nucleotide in the diploid DNA template; and the predicted length of extension of Primer 3 occurring in each of the four different reaction mixes is indicated in the box at the intersection of a column-heading and a row-heading.

FIG. 18, comparable to FIG. 8 except for the use of dNTP-R Bridging Nucleotides instead of dNTP Bridging Nucleotides and omission of Reporting Nucleotide from the four mixes, shows a table in an embodiment of the present invention that can be utilized to determine the identity of an SNP in a haploid DNA template based on incorporation of a labeled Reporting Nucleotide (dTMP-R in this instance) residue, carrying for example a fluorescent dye, non-fluorescent dye or isotopic label, into Primer 3 in the polymerase reaction shown in FIG. 7, without regard to or analysis of the actual length of the primer extension. The headings of columns 2-5 represent different polymerase reaction mixes employed for primer extension; the headings of rows 2-5 in column 6 represent a DNA template sequence with potential permutations of the SNP nucleotide. The predicted amount of label incorporation into Primer 3 occurring in each of the four reaction mixes is indicated in the box at the intersection of a column-heading and a row-heading; wherein 0=no label incorporation, 1×=incorporation of one dNMP-R residues per haploid SNP site, and 3×=incorporation of three dNMP-R residues per haploid SNP site.

FIG. 19, comparable to FIG. 11 except for the use of dNTP-R Bridging Nucleotides instead of dNTP Bridging Nucleotides and omission of Reporting Nucleotide from the four mixes, shows a table in an embodiment of the present invention that can be utilized to determine the identity of an SNP in a diploid DNA template based on incorporation of a labeled Reporting Nucleotide (dTMP-R in this instance) residue into Primer 3 in the polymerase reaction shown in FIG. 7, without regard to or analysis of the actual length of the primer extension; wherein the headings of columns 2-5 represent different polymerase reaction mixes employed for primer extension; the headings of rows 2-11 represent a DNA sequence with potential permutations of the SNP nucleotide in a homozygous or heterozygous diploid DNA template; and the predicted amount of label incorporation into Primer 3 occurring in each of the four reaction mixes is indicated in the box at the intersection of a column-heading and a row-heading; and 0=no label incorporation, 1×=incorporation of one dNMP-R residues per diploid SNP site; 2×=incorporation of two dNMP-R residues per diploid SNP site, 3×=incorporation of three dNMP-R residues per diploid SNP site, and 6×=incorporation of six dTMP-R residues per diploid SNP site.

### DETAILED DESCRIPTION

It will be readily apparent to one skilled in the art that various substitutions and modifications may be made in the invention disclosed herein without departing from the scope and spirit of the invention.

### Terms:

The term “a” or “an” as used herein in the specification may mean one or more. As used herein in the claim(s) the words “a” or “an” may mean one or more than one. As used herein “another” may mean at least a second or more.

The term “Bridging Nucleotide” as used herein refers to a chain-extending deoxyribonucleoside triphosphate, containing a single type of nitrogenous base group (e.g. A, T, G or C), added to a single polymerase reaction mix to enable an extension primer hybridized to a DNA template strand to extend across a target nucleotide (SNP, point mutation or characteristic nucleotide) on the DNA template strand when the added Bridging dNTP is complementary to the target nucleotide.

The term “hybridizing” as used herein refers to a method wherein the association of two complementary nucleic acid strands form double-stranded nucleic acid molecules, which can contain two DNA strands, two RNA strands, or one DNA and one RNA strand. The association of complementary strands occurs under a variety of appropriate conditions (e.g. temperature, pH, salt concentration, etc.) that are well known in the art of molecular biology.

The term “3′ hydroxy terminus”, or more simply “3′ terminus” as used herein refers to the end of a nucleic acid molecule that consists of a sugar molecule with a free, unesterified 3′ hydroxyl group.

The term “Reporting Nucleotide” or “Reporting dNTP-R” as used herein refers to a deoxyribonucleoside triphosphate that can participate in a DNA polymerase reaction bringing about the addition of its deoxyribonucleoside monophosphate (dNMP-R) moiety to the growing 3′ terminus of an extension primer. Both the dNTP-R and the dNMP-R contain a chemical reporter “R” group that can be any measurable group, e.g. any fluorescent dye, non-fluorescent dye or isotopic label covalently attached to the dNTP or dNMP moiety, which does not interfere with the complementary pairing of the dNTP moiety and its participation in DNA polymerization. The “R” group can even be simply a hydrogen atom in the event that mass spectrometry is used to detect or measure the addition of the dNMP-R residue on to the extension primer. When a dNTP-R is complementary to both the target nucleotide and the nucleotide positioned 3′ adjacent to the target nucleotide on the DNA template, the dNTP-R can doubly serve as Bridging Nucleotide and Reporting Nucleotide in a polymerase reaction mix.

The term “5′ adjacent” describing a nucleotide as used herein refers to a nucleotide residue on the DNA template that is positioned immediately adjacent to and on the 5′ side of a reference (or target) nucleotide on a template DNA strand; and the term “3′ adjacent” describing a nucleotide as used herein refers to a nucleotide residue on the DNA template that is positioned immediately adjacent to and on the 3′ side of a reference (or target) nucleotide on a template DNA strand. Thus when an extension primer is hybridized to the template DNA immediately 3′ to the target nucleotide, the growing extension primer elongates by the addition of a dNMP (or dNMP-R) residue derived from a nucleobase that is complementary to the target nucleotide, followed by the addition of a dNMP-R residue that is complementary to the nucleotide on the DNA template that is 5′ adjacent to the target nucleotide on the template.

The term “aliquoting” as used herein refers to dividing a volume uniformly into parts.

The term “incubating” as used herein refers to a favorable environment for processing a reaction mixture. The favorable environment comprises appropriate temperature, enzyme concentration, salt concentration, pH conditions, or other favorable reaction conditions.

The term “polymerase reaction mixture” as used herein refers to favorable components for a DNA polymerase enzyme to extend an oligonucleotide extension primer.

The term “nucleotide” as used herein refers to any compound that consists of a nucleoside esterfied with a phosphate on its sugar moiety.

The term “nucleoside” as used herein refers to a component of a nucleic acid that comprises a nitrogenous base linked to a sugar.

The term “nucleobase” as used herein refers to any nitrogenous base that is a constituent of a nucleoside, nucleotide or nucleic acid, also synonymous with nucleoside base.

One aspect of the present invention relates to a method of identifying or genotyping a target nucleotide, which may be a single nucleotide polymorphism (“SNP”), point mutation or characteristic residue in a nucleic acid molecule. This method utilizes base extensions of oligonucleotidyl extension primers that are complementary to the DNA template molecule containing the target nucleotide. In each instance the predicted extension length of the extension primer in the presence of a Bridging Nucleotide in the form of dNTP or dNTP-R, and in some instances also a Reporting Nucleotide in the form of dNTP-R, where R represents a reporter group that can be a fluorescent dye, non-fluorescent dye or isotopic chemical group, or just a hydrogen atom, covalently bonded to the dNTP moiety of dNTP-R, is compared with the observed extension length to identify the target nucleotide. Accomplishment of the same objective can also be sought by comparing the predicted and observed quantities of “R” label incorporation arising from the incorporation of a labeled dNMP-R residue into the extension primer, where the incorporated label is placed on a non-chain-terminating dNMP-R residue. The current invention thus completely avoids the use of any ddNTP-R that gives rise to the incorporation of a chain-terminating ddNMP-R residue. Instead, incorporation of the dNMP-R residue will not cause chain termination per se; chain termination will occur only when further chain extension calls for reaction between DNA polymerase and a dNTP or dNTP-R nucleotide that is absent from the reaction mixture. The dNTP-R employed here, such as a dye-dNTP, differs from dNTP, the natural substrate of DNA polymerase, by only one count, viz, the presence of the R moiety, and DNA polymerases in general work far better with a non-chain-terminating dNTP-R than they do with a chain-terminating ddNTP or ddNTP-R.

In one embodiment of the present invention an extension primer is hybridized to a nucleic acid template molecule such that the nucleotide on the template complementary to the 3′ terminus nucleotide of the extension primer is 3′ adjacent to the target nucleotide site, i.e. positioned immediately adjacent to and on the 3′ side of the target nucleotide. The extension primer and nucleic acid template are hybridized together to form a hybridized-nucleic acid mixture. The extension primer can then be extended under polymerization conditions that will yield a zero, two-base or longer extension of the extension primer with the complementary nucleic acid bases of the nucleic acid molecule that contains the SNP (or point mutation or characteristic site) acting as template. For example, adding to a DNA polymerase reaction mixture a Reporting Nucleotide dNTP-R that is complementary to the nucleotide on the DNA template that is positioned 5′-adjacent to an SNP site will assure that incorporation of the reporting dNMP-R into the extension primer will occur if the polymerase reaction mixture contains a Bridging dNTP that is complementary to the SNP site nucleotide, so that the chain extension reaction catalyzed by DNA polymerase will cross the gap posed by the SNP site to read the nucleotide 5′ to the SNP site. In this manner, the chain extension reaction will proceed until the reading of a nucleotide on the DNA template calls for reaction with a dNTP that is not included in the polymerase reaction mixture, namely a dNTP species that differs from both the Bridging dNTP and the Reporting dNTP-R. In this embodiment, the same Reporting Nucleotide dNTP-R that is complementary to the nucleotide site immediately 5′ to the SNP is used in each of the four polymerase reaction mixes containing different Bridging Nucleotides. This allows the polymerase reaction to extend the primer by at least one nucleotide past the SNP in the presence of the correct Bridging Nucleotide, which is complementary to the SNP site nucleotide. Experimentally, four reaction mixes are used, all containing the same dNTP-R species. In three of these mixes, a Bridging Nucleotide dNTP is added together with the dNTP-R. In the fourth mix, dNTP-R itself will also serve as the Bridging Nucleotide. The reaction mixes are incubated in the presence of a DNA polymerase for the purpose of extending the 3′ terminus of the extension primer to form an extended primer. The lengths of the extended primer obtained with the four reaction mixes are then determined and compared. The identity of the SNP nucleotide in the nucleic acid template molecule, either located within a haploid gene or a diploid gene, can then be determined by utilizing a table of predicted lengths of the extended primers in each of the four reaction mixes. It can also be determined by utilizing the table of the predicted amounts of label “R” incorporation from a labeled dNTP-R into the primer in each of the four reaction tubes, where the R group is chosen to make possible a quantitation of the amount of its incorporation.

In one embodiment, a one-step primer elongation is followed by analysis of chain length or label “R” incorporation into primer from dNTP-R in order to provide complete information on the SNP nucleotide of interest. In this embodiment, an oligonucleotide primer is furnished having a sequence complementary to the section of the template polynucleotide that is directly adjacent to the SNP nucleotide on the 3′ side. The target nucleotide refers to the position in which the SNP (or point mutation or characteristic residue) to be screened is known to be located on the template. A single Reporting Nucleotide dNTP-R which is complementary to the nucleotide 5′ adjacent to the SNP is also provided in the reaction mixture. The dNTP-R may be in a form where the label “R” is a detectible chemical moiety such as a fluorescent dye, non-fluorescent dye or isotopic group, or simply a hydrogen atom, depending on the method used for product analysis in the subsequent step, e.g. fluorescence detection will require the use of a fluorescent label, whereas detection by mass spectrometry may proceed even with “R” being just a hydrogen atom. Also there may be present in the reaction mix one Bridging Nucleotide or dNTP serving to reveal what kind of Bridging dNTP will make possible the successful extension of the extension primer across the SNP site, thereby revealing the nature of the SNP nucleotide. In the case where the SNP nucleotide and its 5′ adjacent nucleotide on the template DNA share the same nucleobase identity, the Reporting Nucleotide dNTP-R can doubly serve also as a Bridging Nucleotide because in this instance primer extension across the SNP site will occur in the presence of dNTP-R alone without the presence of another Bridging dNTP.

The identity of the nucleotide at the SNP site of the template DNA can be revealed by determining the length of primer extension or the amount of dNMP-R incorporation into primer after the primer extension reaction. Fully informative results can be obtained if different reaction mixes are used, each containing a different Bridging Nucleotide in the form of dNTP. A null reaction that yields no primer extension or label incorporation suggests that the target SNP nucleotide is not complementary to either the Bridging Nucleotide dNTP or the Reporting Nucleotide dNTP-R present in the reaction mix, while a two-base extension suggests that the SNP nucleotide is complementary to the Bridging Nucleotide in the reaction mix. The production of longer than two-base extensions of the extension primer indicates that the nucleotide or nucleotides located at two or more residues 5′ to the SNP site are also complementary to the dNTP or dNTP-R in the mix.

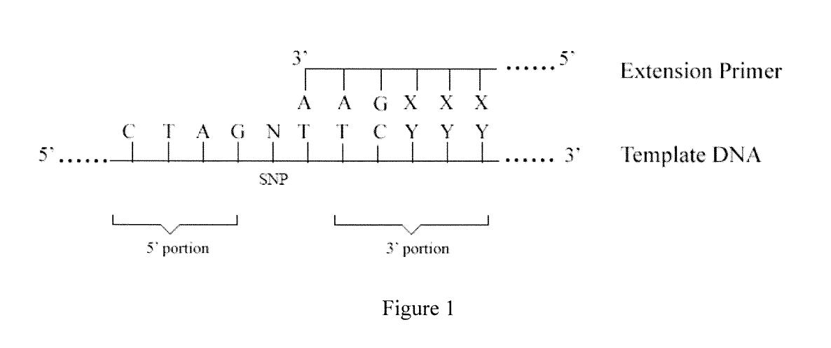

The schematic drawing of FIG. 1 shows one embodiment of the present invention; a strand of template DNA, for example human template DNA, having a 3′ portion and a 5′ portion is hybridized with a complementary oligonucleotide extension primer having a 3′ portion and a 5′ portion; a target nucleotide, (e.g. SNP, point mutation, or characteristic nucleotide) on the template is directly adjacent to the 3′ hydroxy terminus of the extension primer. The DNA template in FIG. 1 shows an SNP with the nucleotide base N, which may be A, T, G or C among human populations. The nucleobases that flank the target nucleotide are known and typically do not vary in frequency in the human populations as does the SNP nucleotide. Thus, for illustration purpose, the three nucleotides on the 3′ side of N are sequentially T. T and C, whereas the four nucleotides on the 5′ side of N are sequentially G, A, T and C. The object will be for a user of this invention to identify the SNP nucleotide N in a given human DNA sample. While the template DNA may be derived from humans, it also may be derived from bacterial and viral nucleic acids, where the target nucleotide could be a characteristic residue which conveys drug resistance to the microorganism, or which distinguishes some strains of a bacterium or virus from other strains.

The following paragraphs describe how a method according to the present invention may be applied to diagnostically identify the target nucleotide at a specified location in an unknown DNA sample. The first step in the process is the purification of the genomic DNA containing the target nucleotide, and its separation from other contaminating materials such as cell debris; such purification methods are well know to individuals with ordinary skill in the art and will not be discussed further here. After DNA purification, an extension primer, as indicated in FIG. 1, is provided for detection purpose according to the present invention. The extension primer contains a sequence that is complementary to the section of the template DNA strand that is directly 3′ to the SNP site N. In this example, for simplicity, only the three bases immediately 3′ adjacent to N are shown. The other bases farther away on the 3′ side are simply indicated as Y's on the template DNA strand, and their complementary bases on the extension primer are indicated as X's. Additional bases on either the template DNA strand or the extension primer are represented simply by a series of dots in the figure. The binding of the extension primer to the template DNA as shown prepares the extension primer to undergo chain extension using the template DNA as template in the presence of DNA polymerase and the necessary dNTP substrates. It is understood that the appropriate reaction conditions have to be provided for chain extension to occur; these conditions are known in the art and may be obtained from standard laboratory manuals such as J. Sambrook, et al. in Molecular Cloning: A Laboratory Manual, Cold Spring Harbor Laboratory Press, 2^{nd} edition, 1989.

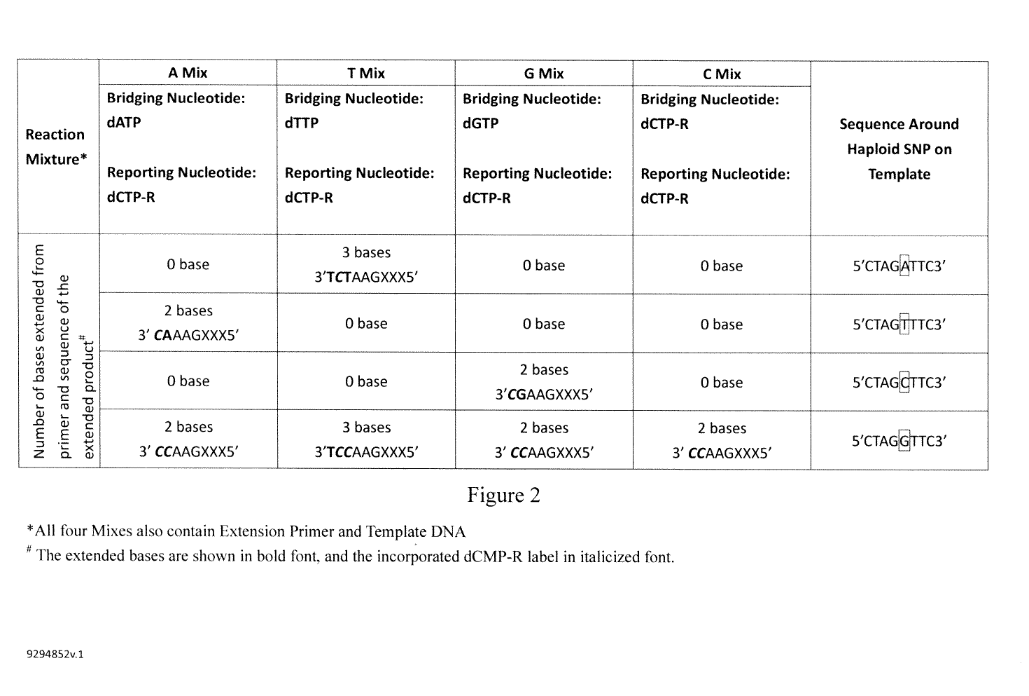

The table of FIG. 2 shows how the identity of the SNP nucleotide N on a haploid DNA template strand can be revealed by chain length analysis of the extension primer upon reaction with DNA polymerase in each of the four polymerase reaction mixes: A Mix, T Mix, G Mix and C Mix, each named according to the nucleobase of the Bridging Nucleotide present in the mix. Each of these four mixes contains the same Reporting Nucleotide dCTP-R plus a Bridging Nucleotide, as well as DNA polymerase and a template DNA hybridized to an extension primer. In this instance, the nucleotide 5′ adjacent to the SNP site being a G, the Reporting Nucleotide selected for its complementarity to the nucleotide at this 5′ adjacent position is accordingly dCTP-R in all four mixes. In this table, the column-headings show the Bridging Nucleotide and the Reporting Nucleotide in the mix, and the row-headings in column 6 show a segment of haploid template DNA sequence with potential permutations of the SNP nucleotide. The predicted length of the extension primer after incubation in each of the four reaction mixes is listed inside the box at an intersection of the column-heading and the row-heading.

In the table in FIG. 2, when the SNP nucleotide is A on the haploid DNA template, e.g. row 2), T Mix containing Bridging Nucleotide dTTP and Reporting Nucleotide dCTP-R will bring about a 3-base chain extension of the extension primer, incorporating sequentially dTMP, dCMP-R, and finally another dTMP; in this instance, extension of a 4^{th} base would require the reading of a T on the template DNA and therefore incorporation of dAMP, which is disallowed because there is no dATP or dATP-R in the T Mix; there is no extension of the extension primer in A Mix, G Mix or C Mix because none of these mixes contains any dTTP or dTTP-R, and the extension primer cannot be extended across the SNP site. When the SNP nucleotide is T (e.g. row 3), the presence of the Bridging Nucleotide dATP in the A Mix, which is complementary to the SNP nucleotide T, allows a 2-base extension of the extension primer incorporating sequentially into the primer dAMP from the Bridging Nucleotide dATP followed by dCMP-R from the Reporting Nucleotide dCTP-R; however, there is no extension in T Mix, G Mix or C Mix because none of these mixes contains any dATP or dATP-R. Likewise, when the SNP nucleotide is C (e.g. row 4), the presence of the Bridging Nucleotide dGTP in the G Mix, which is complementary to the SNP nucleotide C, allows a 2-base extension incorporating dGMP followed by dCMP-R; however, there is no extension in the A Mix, T Mix or C Mix, all of which lack dGTP. When the SNP site is G (e.g. row 5), the presence of the Reporting Nucleotide dCTP-R alone will ensure the incorporation of two successive dCMP-R residues into the extension primer in A Mix, G Mix and C Mix, but the simultaneous presence of dTTP in the T Mix makes possible the reading of the A residue two bases away from the SNP on the 5′ side to bring about the incorporation of an additional dTMP residue following the two successive dCMP-R residues. Because rows 2-5 in the table, representing four possible different SNP site nucleotides in a haploid DNA sample, predict different patterns of extension chain lengths of the extension primer in the four polymerase reaction mixes, it follows that the experimental pattern of extension chain lengths obtained with a given haploid DNA sample will serve to identify the SNP site nucleotide in the sample. The haploid DNA sample can be DNA sample from the human genome (as in the case of X and Y chromosomes in the male), an animal genome, a plant genome, a bacterial genome or a viral genome.

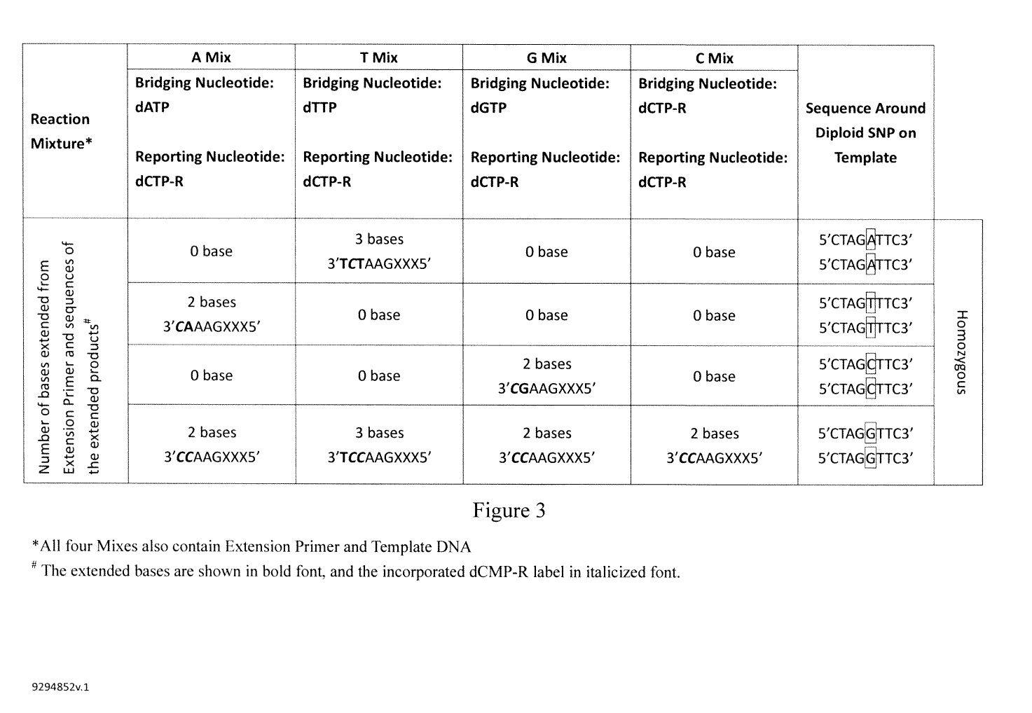

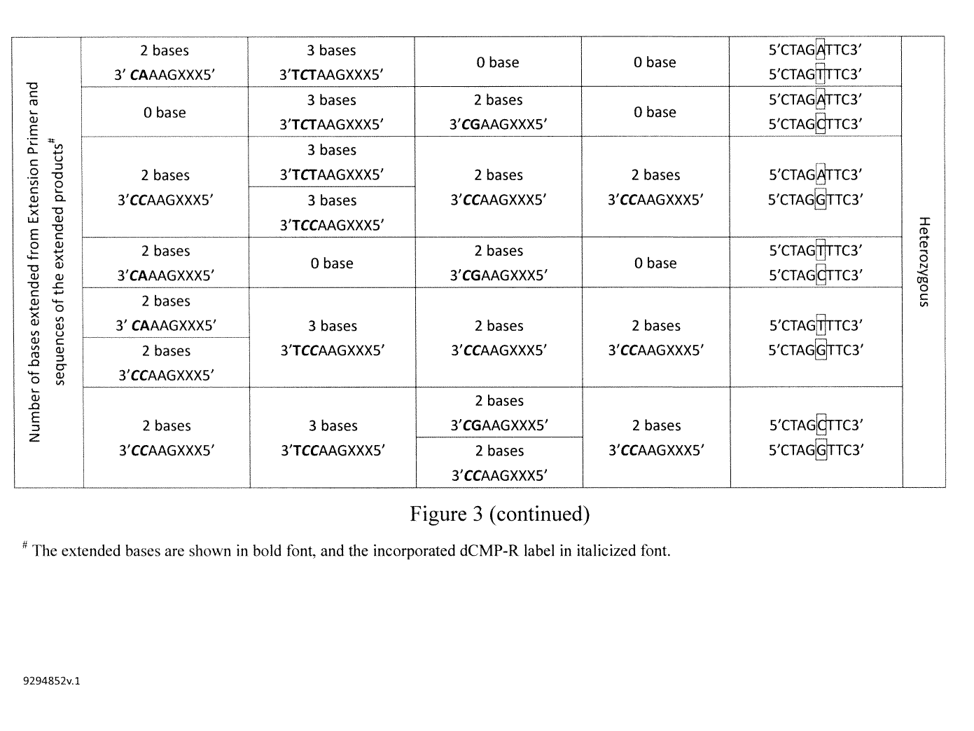

A diploid organism contains two copies of each gene, one from each parent. Genotyping of a diploid organism involves the determination of whether the organism contains two copies of the reference allele (i.e., a reference-type homozygote), one copy each of the reference and variant allele (i.e., a heterozygote), or contains two copies of the variant allele (i.e., a variant-type homozygote). For humans, the genotyping of SNPs can ascertain an individual's susceptibility to a disease, response to a drug or inclination to a personal trait. Individuals that are homozygote for an allele associated with a particular disease are at higher risk of having the disease than a heterozygote or a homozygote for the other allele. The heterozygote, however, is a carrier of the allele associated with the disease. Such knowledge can be useful in prenatal and other types of medical and genetic counseling. When conducting an SNP genotyping analysis, the methods of the present invention can be utilized to interrogate a single target site. Thus the table shown in FIG. 3 can be utilized to determine the nucleotide(s) at an SNP site in a diploid DNA template through chain length analysis of the extension primer following reaction with DNA polymerase in each of four reaction mixes in the reaction system described in FIG. 1. The column-headings in the table indicate the Bridging Nucleotide and the Reporting Nucleotide in the four mixes: the row-headings in column 6 show a segment of diploid template DNA sequence with potential permutations of the SNP nucleotide(s); and the predicted length of extension on the extension primer following incubation with each of the four reaction mixes is listed inside the box at the intersection of a column-heading and a row-heading. For DNA that is homozygous at a given SNP site, namely containing the same SNP nucleotide at the site on both parental copies of the SNP, the predicted extension patterns for the extension primer in each of the four polymerase reaction mixes are given in rows 2-5 of FIG. 3, as in the haploid DNA case in FIG. 2. For example, there is primer extension only in T Mix which contains the A-complementary Bridging Nucleotide dTTP when the SNP site is A e.g. row 2); there is primer extension only in A Mix which contains the T-complementary Bridging Nucleotide dATP when the SNP site is T (e.g. row 3); there is primer extension only in G Mix which contains the C-complementary Bridging Nucleotide dGTP when the SNP site is C (e.g. row 4); and there is primer extension in all four mixes where the common presence of dCTP-R suffices to promote primer extension across the SNP site when the SNP site is G (e.g. row 5). Thus row 2 predicts a 3-base primer extension in T Mix but 0-base extension in A, G and C Mixes, row 3 predicts a 2-base extension in A Mix but 0-base extension in T, G and C Mixes, row 4 predicts a 2-base extension in G Mix but 0-base extension in A, T and C Mixes, and row 5 predicts a 2-base extension in A Mix, G Mix and C Mix but a 3-base extension in T Mix on account of the reading of the A residue two bases away from the SNP on the 5′ side. For each of row 2, row 3 and row 4, the predicted pattern of extensions in the four mixes is unique among rows 2-11. It follows that the observed pattern of extensions suffice to distinguish diagnostically each of the A/A homozygote in row 2, the T/T homozygote in row 3, and the C/C homozygote in row 4 from all nine other homozygous and heterozygous SNP genotypes represented in rows 2-11.

For diploid DNA that is heterozygous at a given SNP site, namely containing different SNP nucleotides occupying the site on the two parental copies of the SNP, the predicted extension patterns for the extension primer in each of the four polymerase reaction mixes are obtained separately for the two parental copies of the SNP, as shown for rows 6-11 of FIG. 3. For example, when the two parental SNP sites are A and T (e.g. row 6), the A Mix allows a 2-base primer extension because it contains Bridging Nucleotide dATP which is complementary to SNP site-T, and Reporting Nucleotide dCTP-R which is complementary to the first nucleobase 5′ to the SNP site; the T Mix allows a 3-base primer extension because it contains dTTP, which is complementary to both the SNP site-A and the second nucleobase 5′ to the SNP site, and Reporting Nucleotide dCTP-R which is complementary to the first nucleobase 5′ to the SNP site. On a similar basis, T Mix allows a 3-base extension, and G Mix allows a 2-base extension in row 7 where an A/C heterotrophy at the SNP site is represented; and both A Mix and G Mix allow a 2-base extension in row 9 where T/C heterotrophy is represented. When the two parental SNP sites are A and G (e.g. row 8), the diploid DNA in the T mix allows two kinds of primer extension: first, extension across SNP site-A to yield a 3-base extension is allowed because T Mix contains the Bridging Nucleotide dTTP which is A-complementary, and extension across SNP site G to yield a 3-base extension is also allowed because T Mix contains the G-complementary Reporting Nucleotide dCTP-R as well, which can doubly serve as Bridging Nucleotide at any SNP site that contains a G residue; in contrast, extension across SNP site G is allowed in Mix A, Mix G and Mix C on account of the presence of dCTP-R in these mixes, but extension across SNP site A is disallowed because these three mixes all lack dTTP.

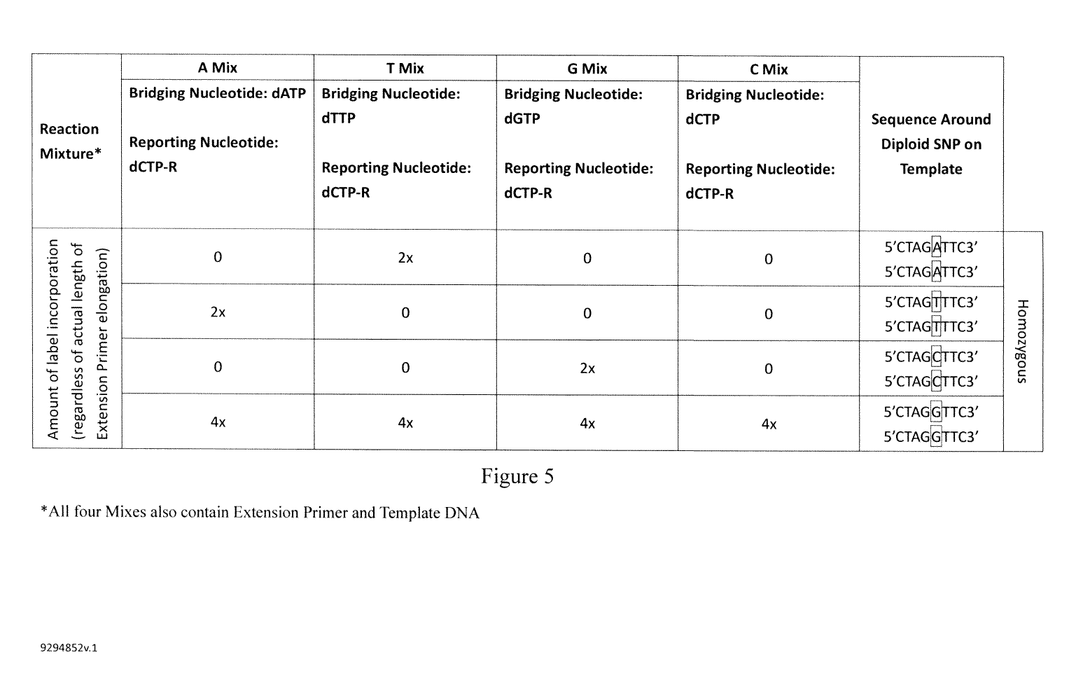

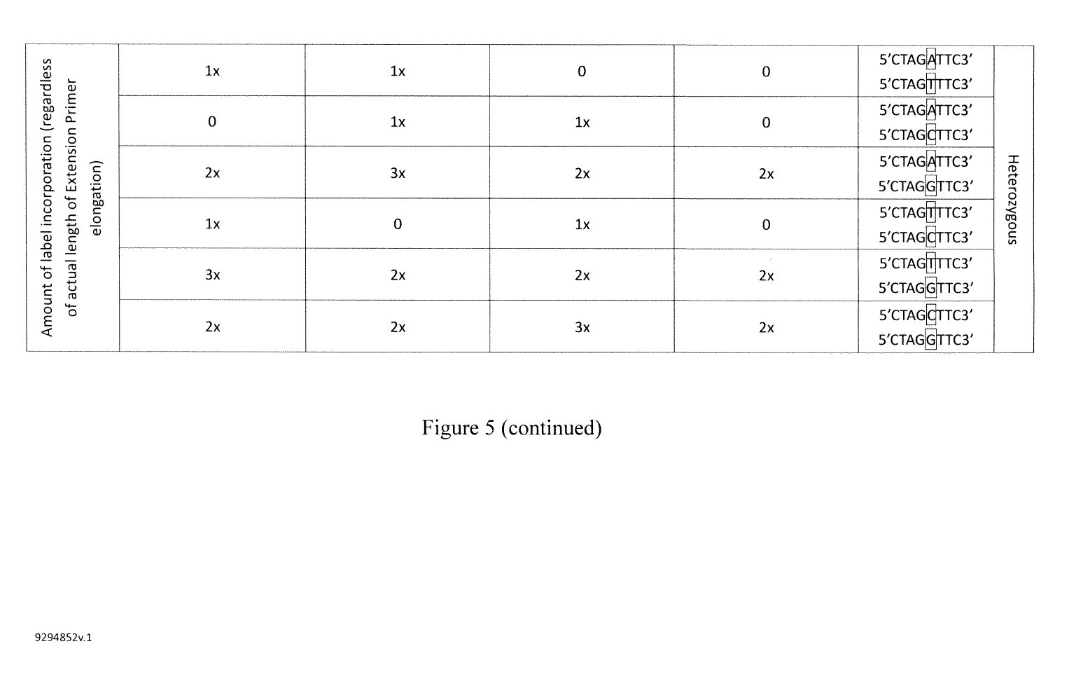

Likewise, when the two parental SNP sites are T and G (e.g. row 10), the diploid DNA sample in the A Mix allows a 2-base extension across SNP site T, as well as a 2-base extension across SNP site G; whereas the other three mixes allow only an extension across SNP site G. Similarly, when the two parental SNP sites are C and G (e.g. row 11), the diploid DNA sample in the G Mix allows a 2-base extension across SNP site C as well as a 2-base extension across SNP site G; whereas the other three mixes only allow an extension across SNP site G. Overall, row 6 predicts a 2-base/3-base/0-base/0-base extension pattern respectively in the A/T/G/C polymerase reaction mixes, whereas row 7 predicts a 0-base/3-base/2-base/0-base extension pattern, and row 9 predicts a 2-base/0-base/2-base/0-base extension pattern. Since each of these three predicted extension patterns is unique among rows 2-11, these predictions suffice to distinguish each of the A/T heterozygote in row 6, the A/C heterozygote in row 7 and the T/C heterozygote in row 9 from all the other homozygous or heterozygous SNP genotypes represented in rows 2-11. In contrast, rows 5, 8, 10 and 11 all predict a 2-base/3-base/2-base/2-base extension profile in the A/T/G/C mixes. Therefore, although the GIG homozygote in row 5, A/G heterozygote in row 8, the T/G heterozygote in row 10 and the C/G heterozygote in row 11 are distinguishable from the other genotypes represented in rows 2-4, 6-7 and 9, they are not differentiated from one another based on the extension-length patterns in the four reaction mixes. However, their diagnostic differentiation from each other can be achieved based on the analysis of label incorporation shown in FIG. 5.

From the examples in FIGS. 2 and 3, it can be clearly seen that, by using a parallel set of four polymerase reaction mixes, the nature of the nucleotide(s) at an SNP site can be identified for a haploid gene, three out of four instances of a diploid gene homozygous with respect to the SNP nucleotide, and three out of six instances of a diploid gene heterozygous with respect to the SNP nucleotide, based on the lengths of primer extensions. The technique used for chain length analysis of the extended primer may be any technique that is available in the art, including electrophoresis or mass spectroscopy. The length of the primer to be employed is dependent on many factors, including the base composition (which affects the melting temperature Tₘ) of the sequence, reaction temperature, hybridization stringency, or other factors as determined by the user. For detection, the Reporting Nucleotide may be unlabelled, using capillary electrophoresis to monitor chain length, or labeled with a fluorophore, dye or radioactive isotope, using fluorometry, colorimetry or radioactive decay for detection.

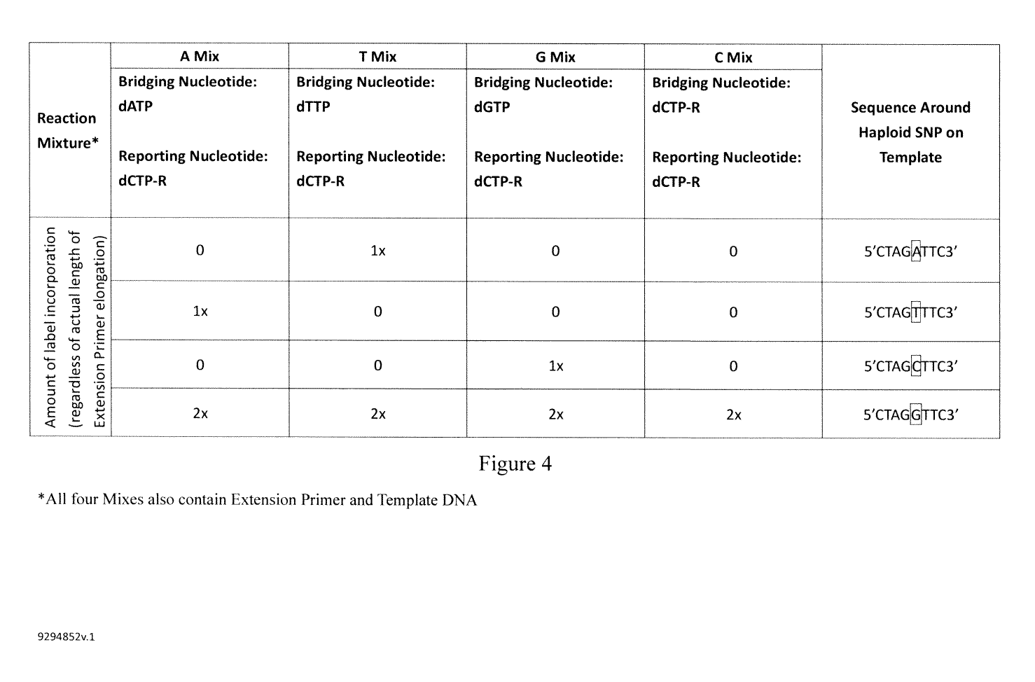

If label measurement employing a labeled reporting dNTP-R is performed, the measurement obtained will indicate the amount of labeled nucleotide incorporation into primer in a given polymerase reaction mixture, regardless of the actual length of primer elongation. Accordingly such measurements provide an alternate basis for determining the identity of an SNP in a DNA sample without regard to or analysis of the actual length of the primer elongation; wherein 0=no label incorporation, 1×=incorporation of one dNMP-R residue, 2×=incorporation of two dNMP-R residues, and 3×=incorporation of three dNMP-R residues, etc. into the extension primer, as illustrated in FIGS. 4-5. In each of these two figures, the column-headings show the Bridging Nucleotide and the labeled Reporting Nucleotide added to the four polymerase reaction mixes, viz. A Mix, T Mix, G Mix and C Mix. The row-headings in column 6 indicate a template DNA sequence with different potential permutations of the SNP nucleotide; and the predicted amount of labeled dNMP-R incorporation into the primer is presented at a column-row intersection where the conditions specified by the pertinent column and row headings apply.

In FIG. 4, the predicted amounts of labeled Reporting Nucleotide incorporated into the extension primer in A Mix, T Mix, G Mix and C Mix for the four different SNP nucleotides at a haploid SNP site follow directly from the extension analysis given in FIG. 2. Thus, when SNP nucleotide is A (e.g. row 2), there is a 3-base primer extension in T Mix including the incorporation into the primer of one dCMP-R residue; accordingly there is “1×” label “R” incorporation into extension primer in T Mix; in contrast, because there is no primer extension in A Mix, G Mix or C Mix, these mixes all show “0” label “R” incorporation. On the same basis, when the SNP nucleotide is T (e.g. row 3), there is “1×” label incorporation in A Mix, but “0” label incorporation in T Mix, G Mix or C Mix. When the SNP nucleotide is C (e.g. row 4), there is “1×” label incorporation in G Mix, but “0” label incorporation in A Mix, T Mix or C Mix. When the SNP nucleotide is G (e.g. row 5), there is “2×” label incorporation in all four mixes, because all the mixes produce a 2- or 3-base extension of the extension primer incorporating two residues of dCMP-R. Since rows 2-5 predict different patterns of label “R” incorporation in the four polymerase mixes, it follows that the experimental pattern of “R” label incorporation obtained with a given haploid DNA sample will serve to identify the SNP site nucleotide in the sample.

For the diploid SNP site shown in FIG. 5, the predicted amounts of labeled Reporting Nucleotide incorporated into the extension primer in A Mix, T Mix, G Mix and C Mix for the four different homozygote and six heterozygote SNP genotypes likewise follow directly from the extension-length analysis given in FIG. 3. When the SNP site is homozygous in A (e.g. row 2), there is no primer extension in A Mix, G Mix or C Mix, and accordingly there is no “R” label incorporation in these three mixes. On the other hand, since there is a 3-base primer extension at both parental SNP sites in the T Mix, in each instance incorporating one dCMP-R residue into the extension primer, it follows that the total number of dCMP-R residue incorporated is “2×” in the T Mix at the diploidal SNP site. When the SNP site is homozygous in T (e.g. row 3), there is no primer extension in T Mix, G Mix or C Mix, and accordingly there is no “R” label incorporation in these three mixes. Since there is a 2-base primer extension at both parental SNP sites in the A Mix, in each instance incorporating one dCMP-R residue into the extension primer, it follows that the total number of dCMP-R residue incorporated is “2×” in the A Mix for the diploidal SNP site. By the same token, when the SNP site is homozygous in C (e.g. row 4), there is no primer extension in A Mix, T Mix or C Mix, and accordingly there is no “R” label incorporation in these three mixes. Since there is a 2-base primer extension at both parental SNP sites in the G Mix, in each instance incorporating one dCMP-R residue into the extension primer, it follows that the total number of dCMP-R residue incorporated is “2×” in the G Mix at the diploidal SNP site. When the SNP site is homozygous in G (e.g. row 5), there is a 2- or 3-base primer extension incorporating two residues of dCMP-R at each of the two parental SNP sites in every mix, and it follows that the total number of dCMP-R residue incorporated is “4×” in each of A Mix, T Mix, G Mix and C Mix.

For the diploid heterozygous SNP sites in FIG. 5, the amounts of labeled Reporting Nucleotide incorporated into the extension primer in A Mix, T Mix, G Mix and C Mix likewise follow directly from the extension-length analysis given in FIG. 3. Thus, since the A/T heterozygote in row 6 in FIG. 3 gives rise to a 2-base primer extension at one of the parental SNP site in the A Mix, and a 3-base primer extension at the other parental SNP site in the T Mix, with incorporation of a single dCMP-R residue in either case, but no extension in G Mix or C Mix, it follows that row 6 of FIG. 5 predicts “1×” incorporation of “R” label in A Mix and in T Mix, and “0” incorporation in G Mix and C Mix. On a similar basis, the A/C heterozygote in row 7 predicts “1×” incorporation in T Mix and in G Mix, but “0” incorporation in A Mix and C Mix; and the T/C heterozygote in row 9 predicts “1×” incorporation in A Mix and in G Mix, but “0” incorporation in T Mix and C Mix. For the A/G heterozygote in row 8, there occurs a primer extension in each of the four mixes at one of the parental SNP sites that results in the incorporation of two dCMP-R residues. In T mix, however, there occurs another mode of primer extension that results in the incorporation of a single dCMP-R residue. Therefore the A/G heterozygote produces a “2×” incorporation in A Mix, G Mix and C Mix, but a “3×” incorporation in T Mix. On a similar basis, the T/G heterozygote in row 10 produces a “2×” incorporation in T Mix, G Mix and C Mix, but a “3×” incorporation in A Mix; whereas the C/G heterozygote in row 11 produces a “2×” incorporation in A Mix, T Mix and C Mix, but a “3×” incorporation in G Mix. Importantly, the four homozygous and six heterozygous SNP sites shown in rows 2-11 of FIG. 5 each displays a unique “R” label incorporation pattern in the four polymerase reaction mixes in terms of the quantified “0”, “1×”, “2×”, “3×” and “4×” incorporations. Consequently, by measuring the quantity of “R” label incorporation in the four mixes, and comparing the measurements to the different predictions given in rows 2-11, the nature of the two parental SNP sites can be elucidated.

To facilitate the experimental distinction between 0, 1×, 2×, 3× and 4× label incorporations in FIGS. 4 and 5, standard SNP-containing DNA templates predicting 0, 1×, 2×, 3× and 4× incorporations may be included in each experimental screening alongside unknown SNP-containing DNA-templates. Since the SNP nucleotides in these standard DNA templates are already known from prior sequence determination, the dNMP-R incorporations they display will provide useful quantitative measures for calibrating the amounts of dNMP-R incorporations observed with the unknown DNA templates. It is also noteworthy that for every SNP-site containing the DNA-strand sequence shown in FIGS. 4 and 5, its complementary DNA-strand will also contain a SNP site that will also be usable for the purpose of genotyping the SNP. For example, in row 11 of FIG. 5, the C/G heterozygous sequences are 5′CTAG[C]TTC3′ and 5′CTAG[G]TTC3′, where the SNP nucleotide is boxed inside [ ]. The sequences on their complementary strands will be 5′GAA[G]CTAG3′ and 5′GAA[C]CTAG3′. It is entirely feasible to analyze these two latter sequences instead of the two sequences given in row 11 of FIG. 5. In so doing, and employing dTTP-R as Reporting Nucleotide in the four different polymerase reaction mixes, these two latter sequences will predict “0” incorporation of dTMP-R into extension primer in the A Mix and T Mix, but “2×” incorporation of dTMP-R in each of G-Mix and C-Mix, because in these instances extension primer elongation, once it crosses the SNP site, will terminate only after incorporation of two successive dTMP-R residues; this 0-base/0-base/2-base/2-base pattern predicted for the A/T/G/C Mixes is much easier to test compared to the corresponding 2-base/2-base/3-base/2-base pattern predicted for the A/T/G/C Mixes in row 11 of FIG. 5. Thus, in this example, the task of identifying the heterozygous diploid SNP nucleotides is greatly facilitated by choosing the 5′GAA[G]CTAG3′ and 5′GAA[C]CTAG3′ template sequences as target of genotyping instead of the 5′CTAG[C]TTC3′ and 5′CTAG[G]TTC3′ sequences shown in row 11 of FIG. 5.

While FIGS. 1-5 show embodiments of the present invention for SNP genotyping using a sample of natural DNA, exactly the same methodology is applicable to DNA that is amplified by polymerase chain reaction (PCR) in other embodiments of the present invention. Using PCR, a very small quantity of double stranded genomic DNA sample is isolated from a subject, and subjected to polymerase chain reaction to amplify the DNA sample. By vastly reducing the quantity of DNA sample that needs to be isolated from a subject, this will substantially enhance the usefulness of the present invention.

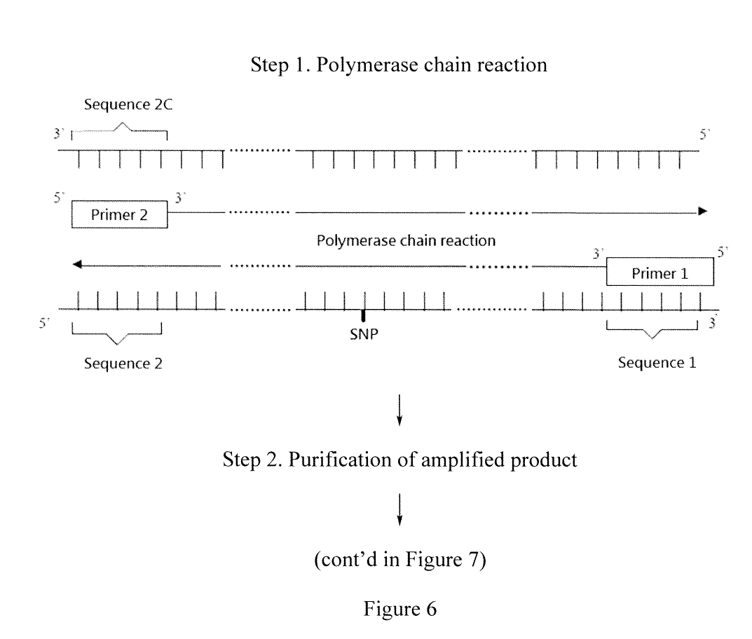

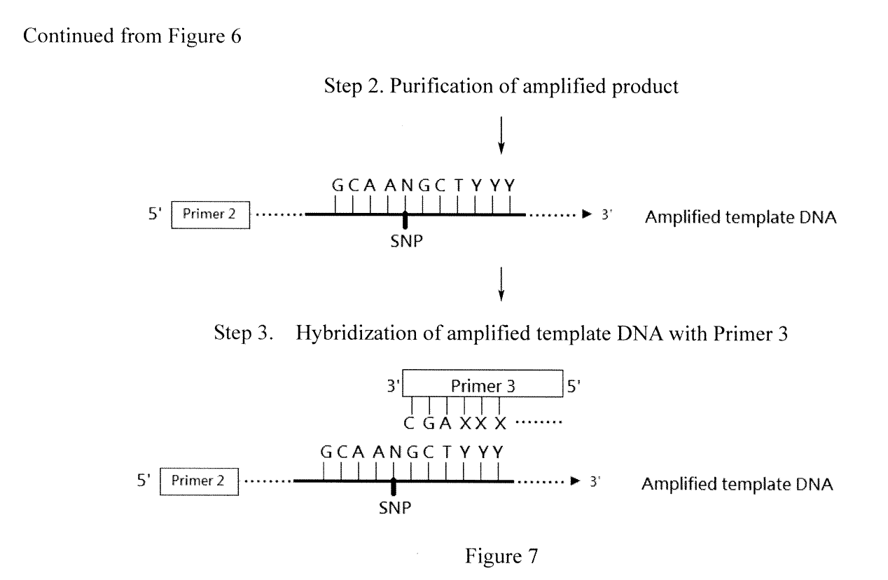

The procedure described in FIGS. 6 and 7 is similar to that described in FIG. 1 except that PCR amplification is used before the polymerase extension reaction is carried out for SNP determination. In this instance, a DNA molecule is subjected to PCR using the oligonucleotidyl PCR Primers 1 and 2 to amplify a selected portion of the DNA molecule containing a SNP or target nucleotide interposed between Primers 1 and 2. The amplified double-stranded DNA product is purified from the unreacted single-stranded Primers 1 and 2 using conventional methods such as size exclusion chromatography. The amplified product also may be freed of amplification primers and dNTPs by for instance digestion with Exonuclease I (ExoI) and shrimp alkaline phosphatase.

In FIG. 7, the amplified DNA strand containing a target SNP site (also referred to as the amplified template DNA) is hybridized with a third complementary oligonucleotide Primer 3 having a 3′ portion and a 5′ portion; the target SNP nucleotide N is directly 5′ adjacent to the G residue that is complementary to the C residue at the 3′ hydroxy terminus of Primer 3. Primer 3 acts as an extension primer, and is therefore analogous to the extension primer in FIG. 1. A,T,C,G are the four standard DNA nucleotides, whereas X and Y, while consisting of also A, T, C or G, are specified only as being complementary with one another on the opposing strands of the double-stranded DNA formed between amplified template DNA and Primer 3.

When DNA polymerase, a Bridging Nucleotide and a Reporting Nucleotide (dNTP-R) are added to the hybridized amplified template DNA and Primer 3 shown in Step 3 of FIG. 7, Primer 3 will undergo DNA polymerase-catalyzed chain extension across the SNP site provided that the Bridging Nucleotide added is complementary to the SNP nucleotide; the extension will continue leading to incorporation of the dNMP-R residue from the Reporting Nucleotide because in the present method the Reporting Nucleotide is always selected to be complementary to the nucleotide on amplified template DNA that is immediately 5′ to the SNP site. For the system depicted in FIG. 7, this nucleotide immediately 5′ adjacent to the SNP site is A; therefore the selected Reporting Nucleotide is dTTP-R.

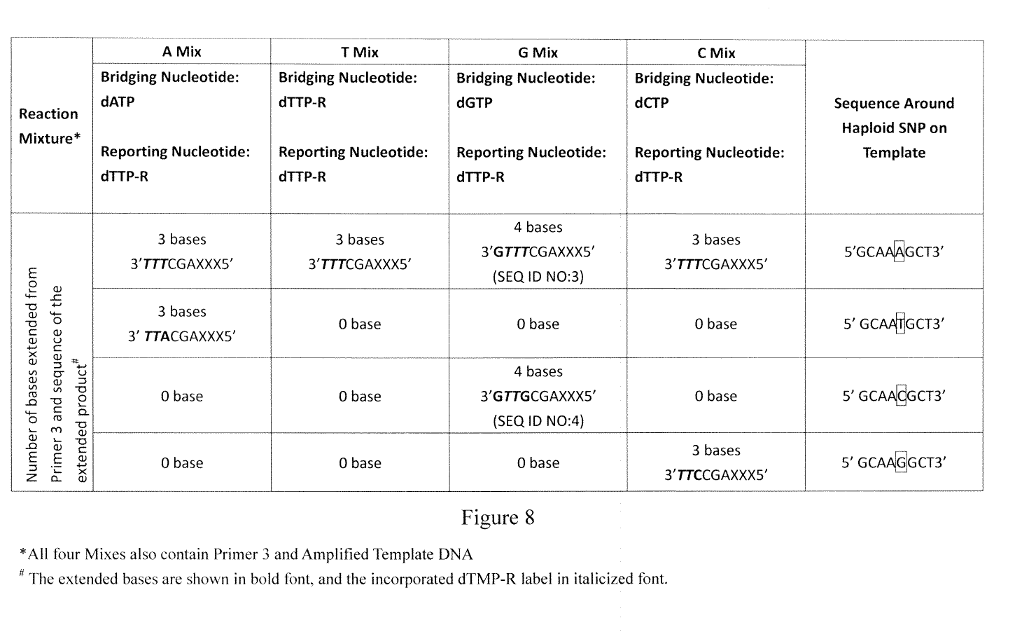

To illustrate how the present invention can be used to identify the SNP nucleotide in a PCR-amplified haploid template DNA sequence as represented in FIG. 7 based on DNA lengths of polymerase-catalyzed primer extensions, FIG. 8 shows the expected results for the set of four reaction mixes, namely A Mix, T Mix, G Mix and C Mix containing respectively the Bridging Nucleotide dATP, dTTP-R, dGTP and dCTP. The Reporting Nucleotide is dTTP-R in all four mixes. Because dTTP-R is the selected Reporting Nucleotide, it doubly serves as Bridging Nucleotide as well in the T Mix. The lengths of Primer-3 extensions predicted by the four possible SNP site nucleotides are arrived at and shown in rows 2-5 of FIG. 8 as in the example of extension primer extensions in FIG. 2. In row 2 where the SNP nucleotide is A, the Reporting Nucleotide dTTP-R is complementary to the SNP nucleotide as well as the two A residues immediately 5′ adjacent to the SNP site on the amplified template DNA; the presence of dTTP-R in all four mixes thus allows Primer 3 to extend across the SNP site as well as the two A-residues 5′ adjacent to the SNP site in all four mixes. In the G Mix, the presence of the Bridging Nucleotide dGTP further enables extension over C, the third residue 5′ to SNP site; thereafter extension stops because the absence of dCTP from this reaction mix precludes extension over G, the fourth residue 5′ to the SNP site. In rows 3, 4 or 5, where the SNP nucleotide is T, C or G respectively, Primer 3 undergoes extension only in a single reaction mix in each row, giving rise to a 3-base extension only in Mix A in row 3, a 4-base extension only in Mix G in row 4, and a 3-base extension only in C Mix in row 5. Notably, because rows 2-5 in the table, representing four possible different SNP site nucleotides in an amplified haploid DNA sample, predict different patterns of extension chain lengths of Primer 3 in the four polymerase reaction mixes, it follows that the experimental pattern of extension chain lengths obtained with a given haploid DNA sample will serve to identify the SNP site nucleotide in the sample. The haploid DNA sample again can be DNA sample from the human genome, an animal genome, a plant genome, a bacterial genome or a viral genome.

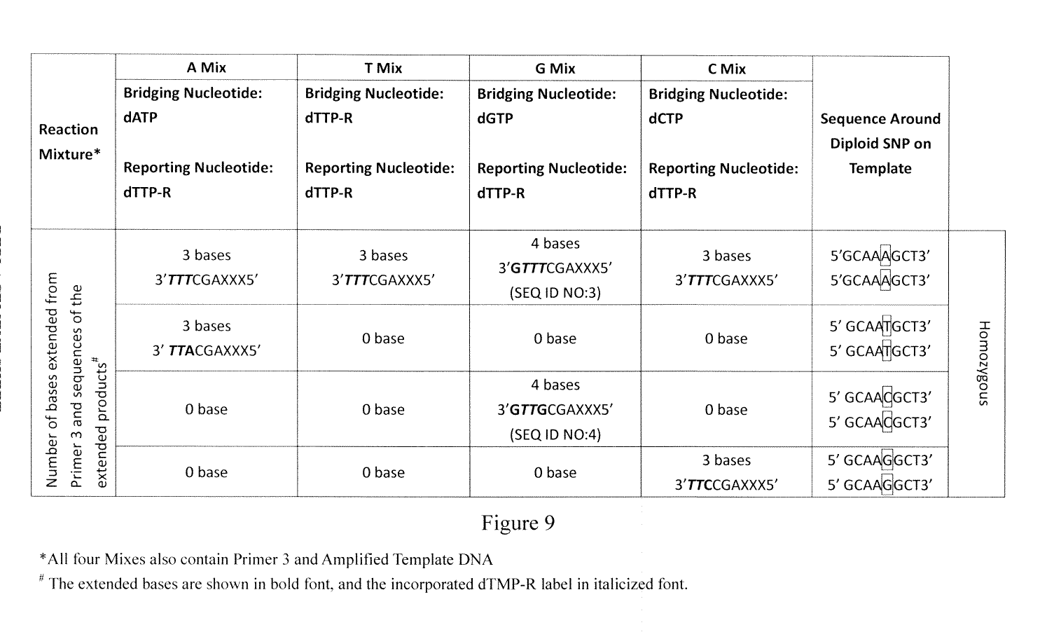

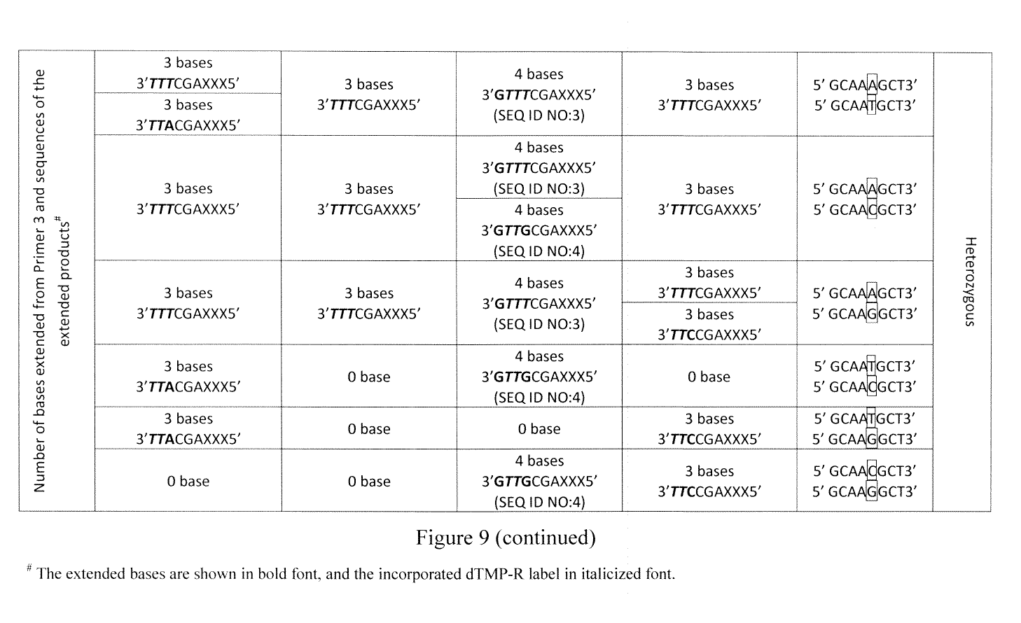

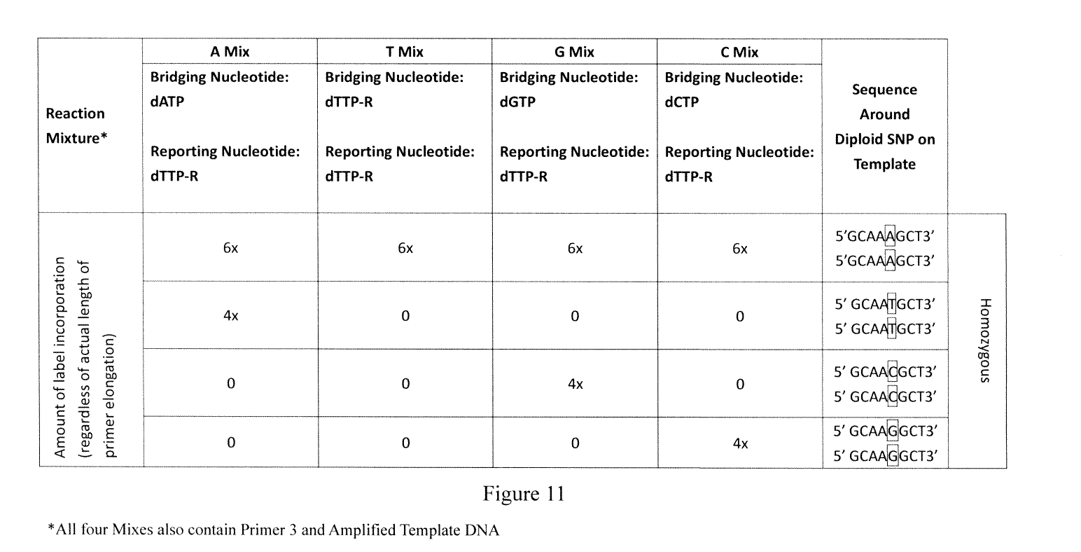

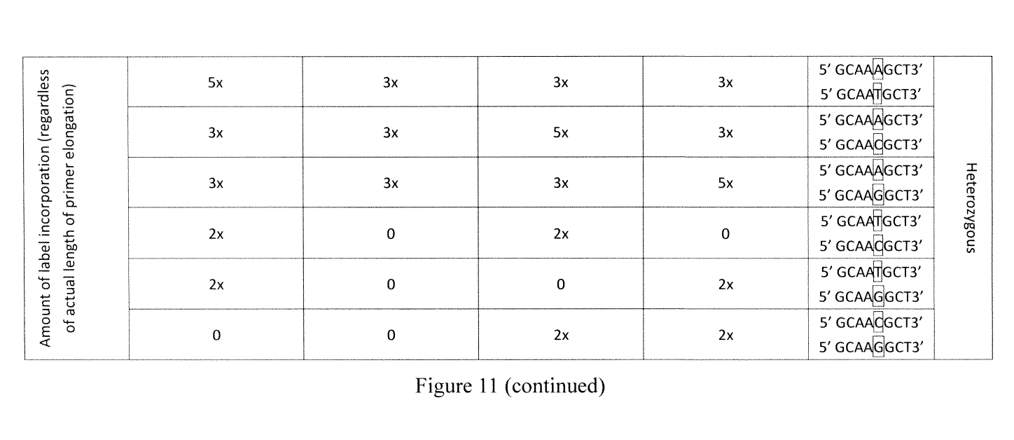

For a PCR-amplified diploid template DNA sequences as represented in FIG. 7, FIG. 9 shows the expected primer extension results in the four different polymerase reaction mixes. The lengths of Primer 3 extension predicted for the four homozygous diploid SNP sites and six possible heterozygous SNP sites are indicated in rows 2-11 of FIG. 9. The predictions of the four homozygous diploid cases (rows 2-5) are entirely similar to those for the haploid SNP site analyzed in FIG. 8. The predictions of Primer 3 extensions in the four reaction mixes for the six heterozygous diploid SNP sites fall into three classes. First, where neither of the diploidal templates support any Primer 3 extension across the SNP site, “0” extension is predicted. Secondly, where only one of the diploidal templates supports Primer 3 extension across the SNP site, the prediction is arrived at as in the case of haploidal templates. Thirdly, when both of the diploidal templates support Primer 3 extension across the SNP site, there will be two extension products within the same polymerase reaction mix; and the predicted extensions are simply the sum of two individual Primer 3 extensions, one for each diploidal template. Among rows 2-11 of FIG. 9, row 3 predicts a 3-base/0-base/0-base/0-base Primer 3 extension pattern for the A/T/G/C Mixes respectively; this pattern is unique to row 3, shared by no other row in the figure. Likewise, each of rows 4-5, and 9-11 predicts a unique Primer 3 extension pattern shared by no other row in the table. It follows that the Primer 3 extension pattern suffices to identify each of the potential T/T, C/C, G/G, T/C, T/G and C/G SNP genotypes in the amplified template DNA. This leaves the 3-base/3-base/4-base/3-base extension patterns of rows 2 and 6-8 in the four mixes undifferentiated from one another. However, the diagnostic differentiation of the A/A, A/T, A/C and A/G SNP genotypes in these rows from one another can be achieved based on the analysis of label incorporation shown in FIG. 11.

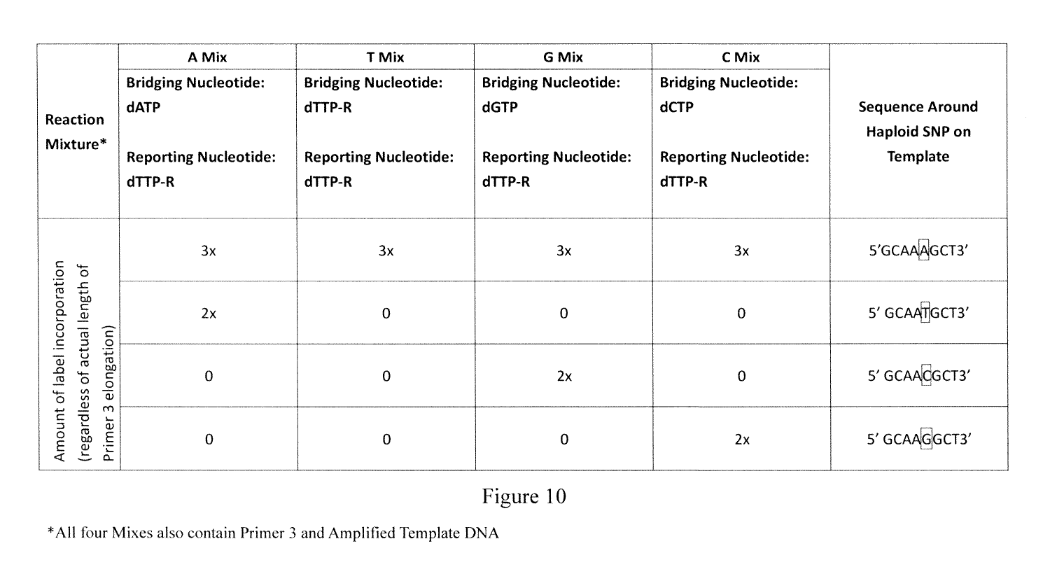

For the PCR-amplified haploid template DNA sequences represented in FIG. 7 containing an SNP site, the nature of the SNP nucleotide at the SNP site can also be diagnostically determined by measuring the amount of incorporation into Primer 3 of labeled nucleotide derived from the Reporting Nucleotide, instead of measuring the lengths of Primer 3 extensions. Rows 2-5 in FIG. 10 show the predicted amounts of incorporation of labeled dTMP-R residues derived from the Reporting Nucleotide dTTP-R in the four different reaction mixes for different SNP nucleotides. These predictions follow directly from the predicted Primer 3 extension products indicated in FIG. 8. For example, for SNP nucleotide A, FIG. 8 shows a 3-base extension in each of A Mix, T Mix and C Mix, and a 4-base extension in G Mix, in all four instances leading to the successive incorporations of three dTMP-R residues; thus the amount of label incorporation predicted in FIG. 10 is “3×” for all four mixes. For SNP nucleotide T, FIG. 8 shows 0-base extension in T Mix, G Mix and C Mix, but a 3-base extension incorporating two dTMP-R residues in A Mix; therefore a “2×” incorporation in A Mix but “0” incorporation in T Mix, G Mix and C Mix are predicted. On a comparable basis, SNP nucleotide C predicts “2×” incorporation in G Mix but “0” incorporation in A Mix, T Mix and C Mix; and SNP nucleotide G predicts “2×” incorporation in C Mix but “0” incorporation in A Mix. T Mix and G Mix. Since rows 2-5 in FIG. 10 predict different patterns of label “R” incorporation in the four polymerase mixes, it follows that the experimental pattern of “R” label incorporation obtained with a given haploid DNA sample will serve to identify the SNP nucleotide in the sample.

For the PCR-amplified diploid template DNA sequences represented in FIG. 7, rows 2-5 in FIG. 11 show the amounts of incorporation of labeled dTMP-R residues from the Reporting Nucleotide dTTP-R in the four different reaction mixes for different diploid homozygous SNP genotypes. These predictions are identical to those shown for the corresponding haploid genotypes shown in rows 2-5 of FIG. 10, except that the number of “R” labels incorporated per diploid SNP site are twice the number of “R” labels incorporated per haploid SNP site. With the different heterozygous genotypes depicted in rows 6-11, the predicted amounts of “R” label incorporations again follow directly from the predicted Primer 3 extension products indicated in rows 6-11 in FIG. 9: for any heterozygous genotype, the amount of “R” incorporation in a particular reaction mix is directly given by the total number of dTMP-R residues incorporated in the Primer 3 extension products indicated in FIG. 9 for the indicated genotype and reaction mix. Wherever a given heterozygous genotype predicts the formation of two kinds of Primer-3 extension products, summation of the dTMP-P residues 1 incorporated into both extension products yields the total incorporation of the dTMP-P “R” label. Notably, because each of rows 2-11 in FIG. 11 predicts a unique A/T/G/C Mix profile of “R” label incorporations into Primer 3, it follows that the amount of “R” label incorporation measured experimentally will suffice to identify the SNP nucleotide(s) in the amplified template DNA, regardless of the SNP site being a homozygous or heterozygous diploidal site.

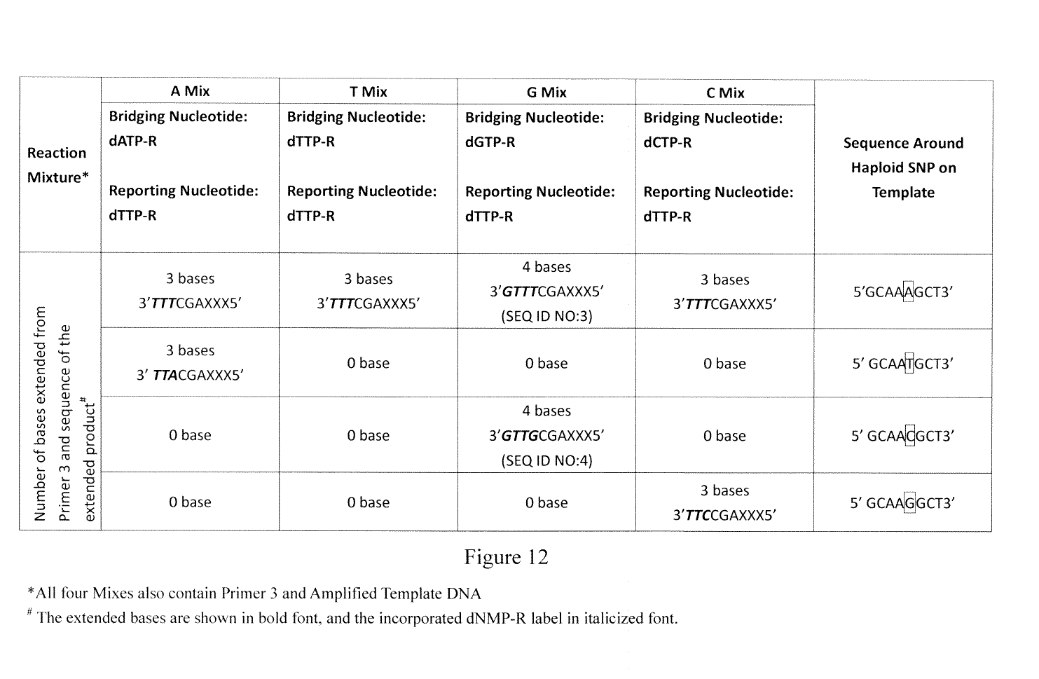

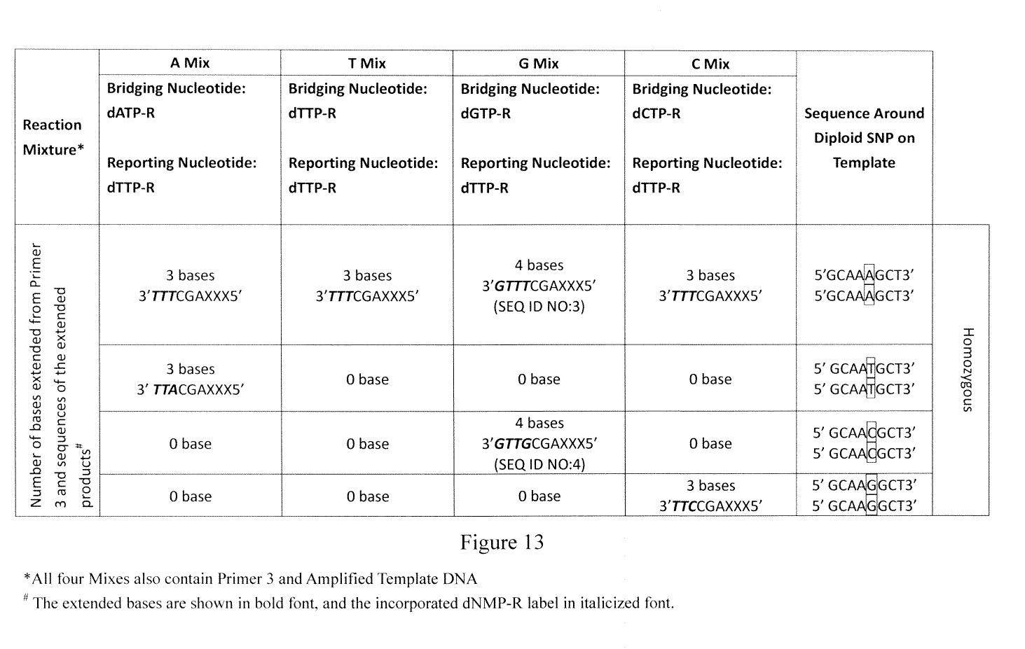

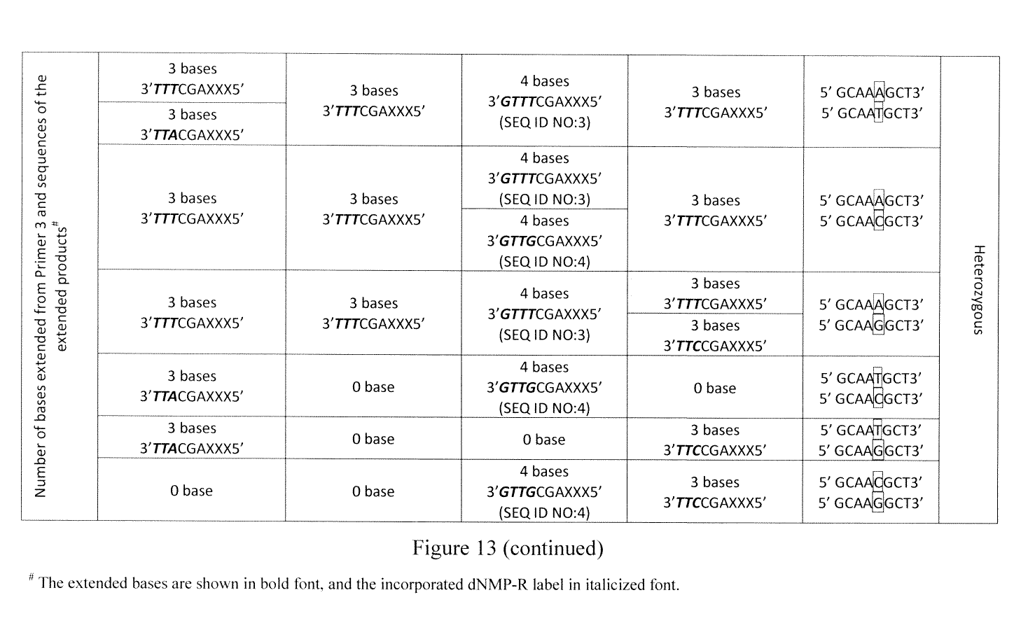

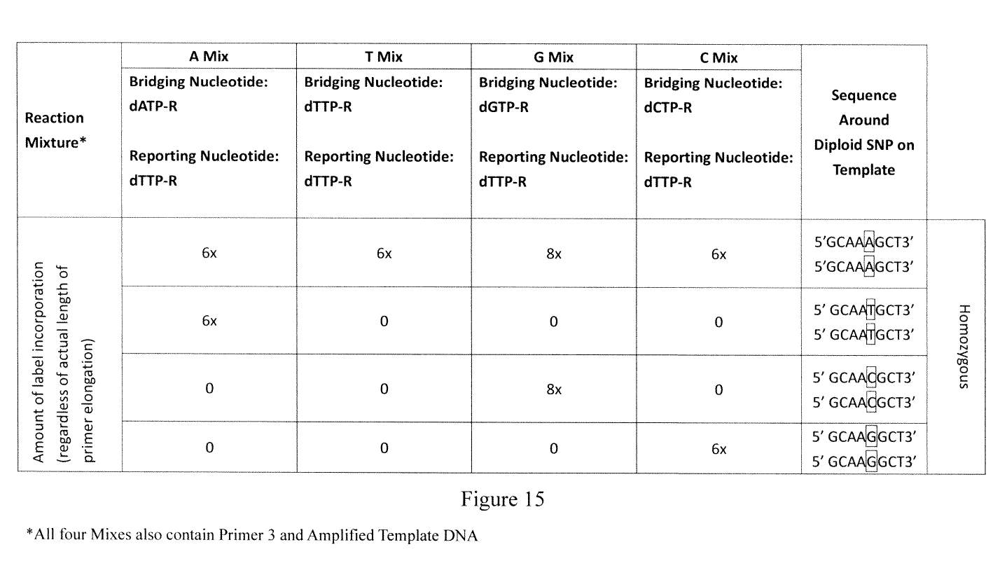

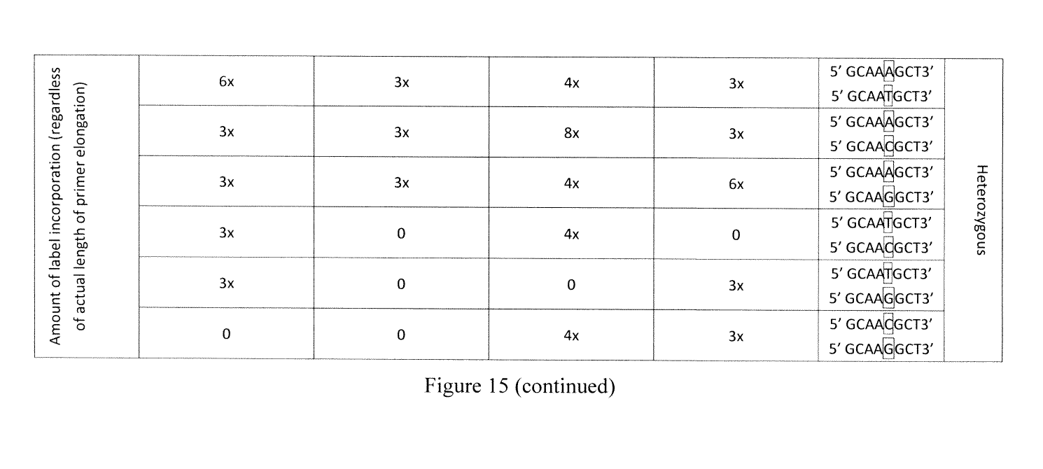

FIGS. 8-11 describe the genotyping of the SNP site shown in FIG. 7 based on primer extension analyzed by either primer chain lengths or label incorporation into primer in A Mix, T Mix, G Mix and C Mix, each containing a Bridging Nucleotide dNTP and a Reporting Nucleotide dNTP-R, or a Reporting Nucleotide dNTP-R that also serves as a Bridging Nucleotide. R represents a chemical label group that can be a fluorescent dye, non-fluorescent dye, isotopic label or simply a hydrogen atom. Because the Bridging Nucleotides and Reporting Nucleotide employed do not include any chain-terminating nucleotide, in another embodiment of the present invention one may employ different labeled dNTP-R Bridging Nucleotides instead of unlabeled dNTP Bridging Nucleotides in the reaction mixes together with a labeled Reporting Nucleotide. This is represented in FIGS. 12-15, where both the Bridging Nucleotides and the Reporting Nucleotide contain the same chemical label R, thereby avoiding any grossly unequal polymerase reaction rates arising from the use of two or more different chemical labels on these nucleotides. FIGS. 12 and 13 using labeled Bridging Nucleotides are entirely similar to FIG. 8 and FIG. 9 using unlabeled Bridging Nucleotides in terms of the primer chain lengths predicted for the A, T, G and C Mixes. Thus in both FIG. 12 and FIG. 8, the chain lengths predicted in rows 2-5 make possible the identification of a haploidal SNP site based on the chain lengths of the extended primer in the four mixes. For a diploidal SNP site, the chain lengths predicted in rows 2-11 of FIG. 13, as in the case of FIG. 9, make possible the identification of the T/T, C/C, GG. T/C, T/G and C/G genotypes, each of which predicts a unique extension pattern in the A/T/G/C Mixes, but cannot differentiate between the A/A, A/T. A/C and A/G genotypes, all of which predict a 3-base/3-base/4-base/3-base extension pattern in the A/T/G/C Mixes. Instead, differentiation between the latter four genotypes can be obtained based on the predicted label incorporations in FIG. 15.

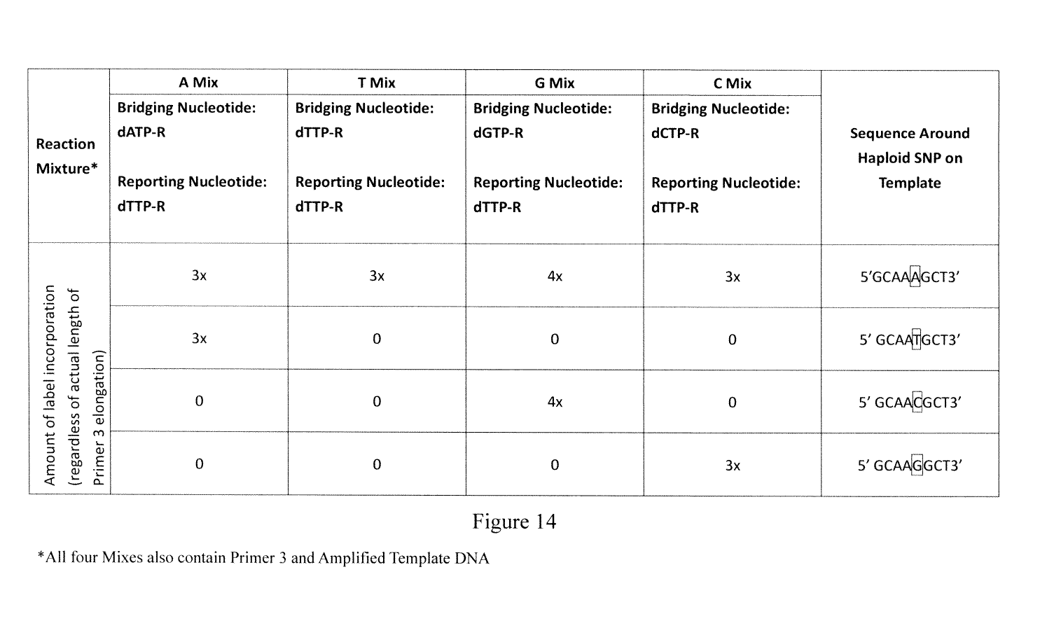

FIG. 14 analyzes label incorporation for a haploidal SNP, and FIG. 15 analyzes label incorporation for a diploidal SNP, for the SNP site shown in FIG. 7 using labeled Bridging Nucleotides together with labeled Reporting Nucleotide. Since the predicted label incorporations in the A/T/G/C Mixes are different in rows 2-5 in FIG. 14, these predictions make possible the identification of each of the haploidal A, T, G and C genotypes, as in the case of FIG. 10. Furthermore, since the predicted label incorporations in the A/T/G/C Mixes are all different in rows 2-11 in FIG. 15, these predictions make possible the identification of each of the four homozygous and six heterozygous diploid genotypes, as in the case of FIG. 11. However, in FIG. 11, the differentiation between the A/T, A/C and A/G genotypes depends on the experimental distinction between the 5×/3×/3×/3×, 3×/3×/5×/3× and 3×/3×/3×/5× patterns of label incorporation in the A/T/G/C Mixes. In contrast, in FIG. 15, the differentiation between the A/T, A/C and A/G genotypes depends on the experimental distinction between the 6×/3×/4×/3×, 3×/3×/8×/3× and 3×/3×/4×/6× patterns of label incorporation in the A/T/G/C Mixes. Since a 3× versus 6×, or 3× versus 8×, distinction is more reliably detected than a 3× versus 5× distinction, FIG. 15 usefully provides an improved basis over FIG. 11 for differentiation between the A/T, A/C and A/G genotypes.

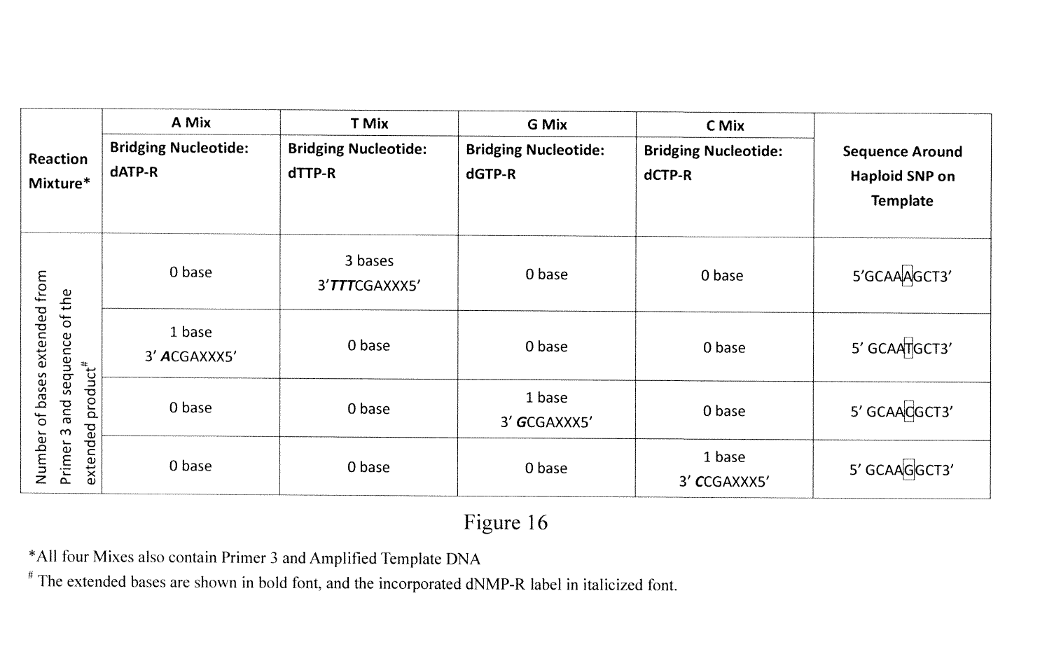

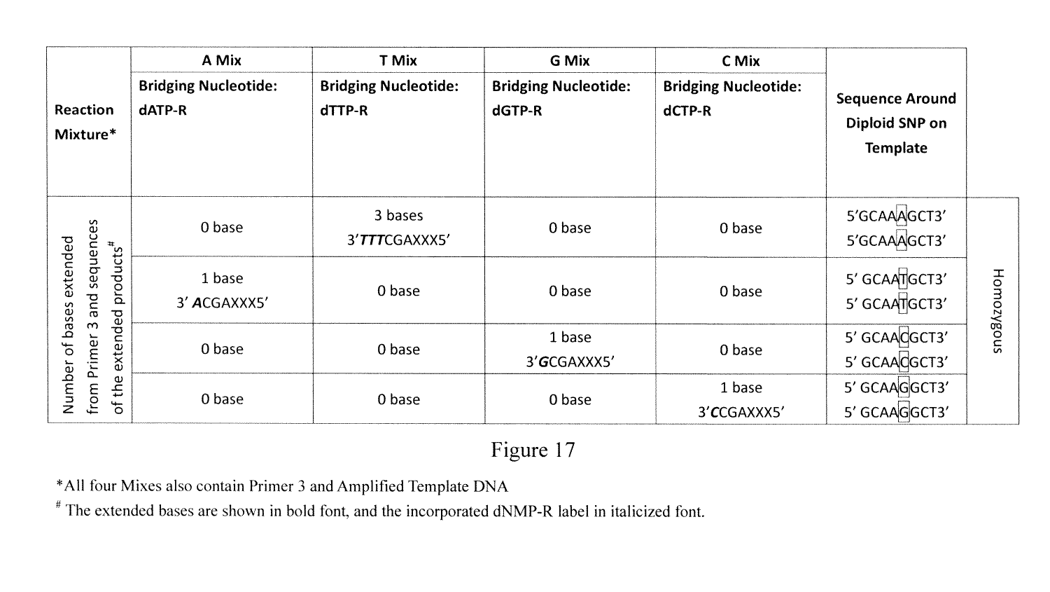

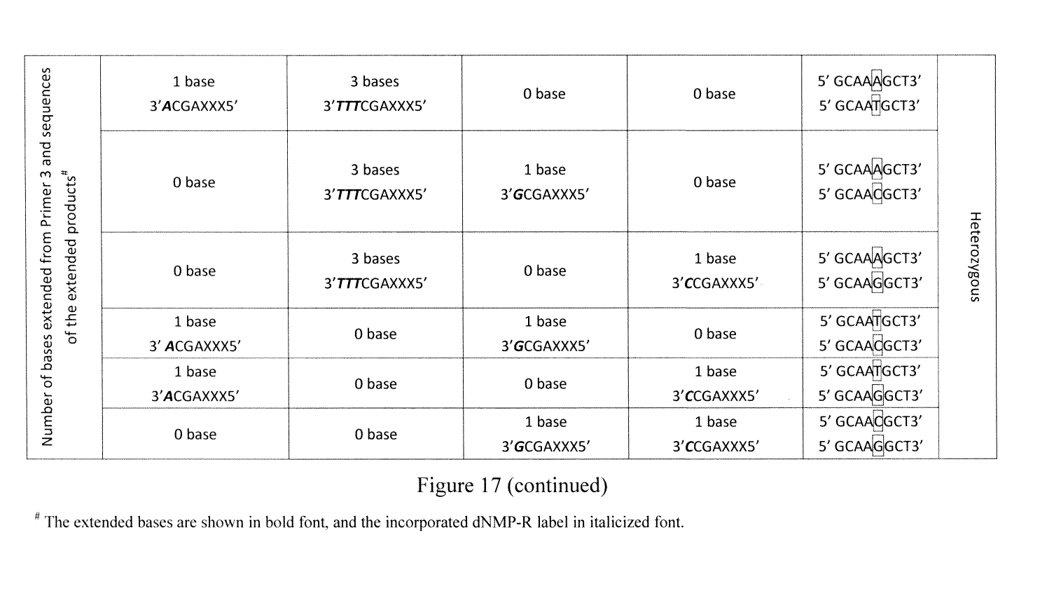

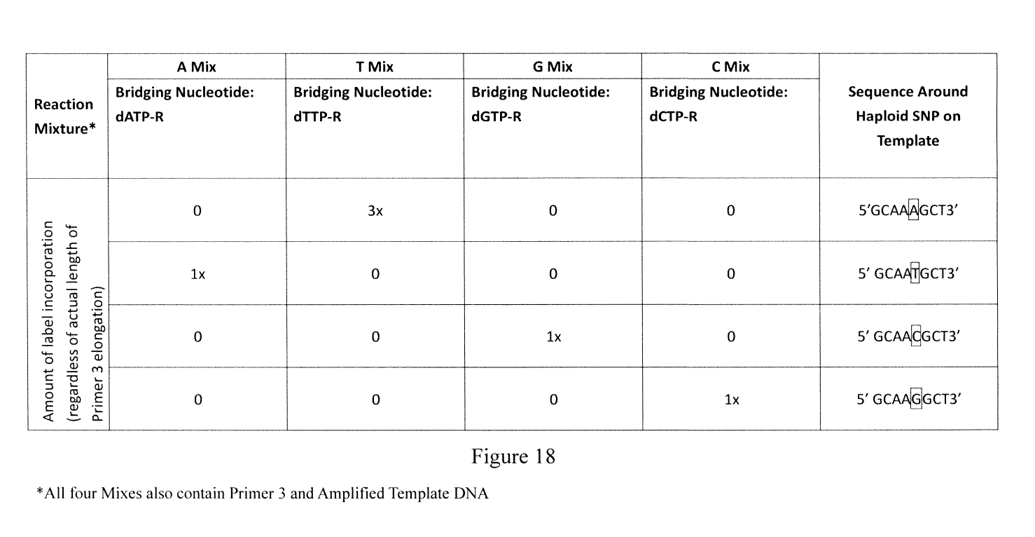

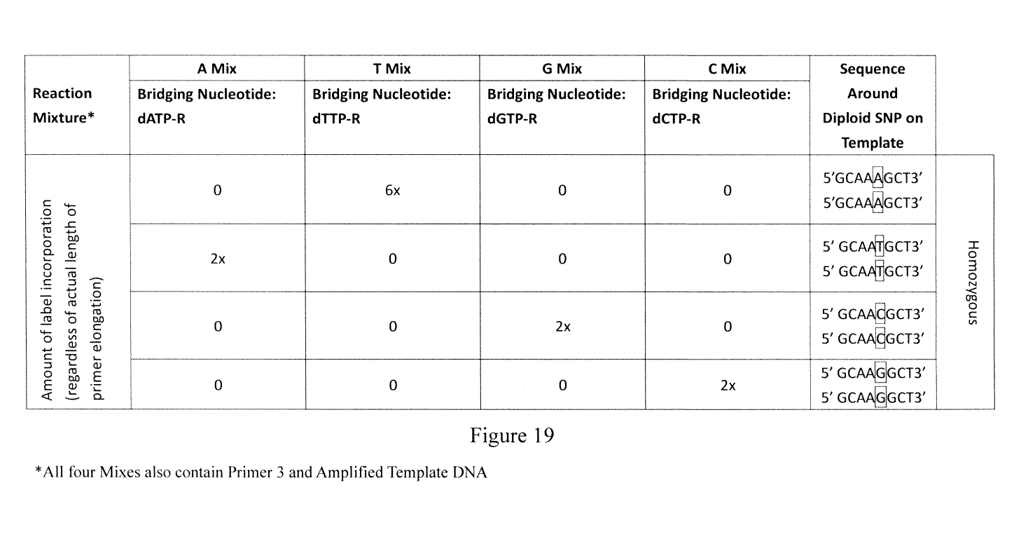

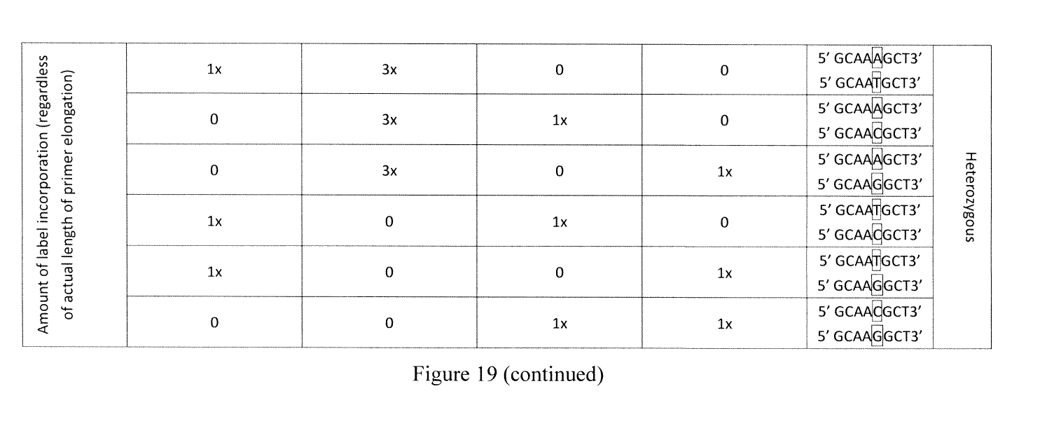

In FIGS. 12-15, the A, T, G and C Mixes each contains a distinct Bridging Nucleotide and a common Reporting Nucleotide, both of which are non-chain-terminating dNTP-Rs, and both of which are capable of reporting on the composition of the extended extension primer. This renders the presence of the common Reporting Nucleotide in these reaction mixes non-essential. Accordingly, in still another embodiment of the present invention based on primer extension with solely non-chain-terminating nucleotides, the A, T, G and C Mixes in FIGS. 12-15 may be modified by omitting the Reporting Nucleotide that is common to the four mixes, leaving in each of the four mixes only a mix-specific Bridging Nucleotide along with Amplified Template DNA and DNA polymerase. This embodiment is shown in FIGS. 16 and 17, which examine the chain lengths of the extended primer in the four mixes for a haploid and a diploid SNP respectively, and in FIGS. 18 and 19 which examine the amounts of incorporation of dNMP-R into the extended primer in the four mixes for a haploid and a diploid SNP respectively. Since rows 2-5 each predict a unique pattern of primer extension in the A/T/G/C Mixes in FIG. 16, the primer extension predictions in this figure make possible the identification of each of the A, T, C and G haploid genotypes. Likewise, since rows 2-11 each predict a unique pattern of primer extension in the A/T/G/C Mixes in FIG. 17, the primer extension predictions in this figure make possible the identification of each of the A/A, T/T, C/C and G/G homozygous diploid genotypes, as well as each of the A/T, A/C, A/G, T/C, T/G and C/G heterozygous diploid genotypes.

Since rows 2-5 each predict a unique pattern of incorporation of the dNMP-R label in the A/T/G/C Mixes in FIG. 18, the amounts of label incorporation predicted in this figure make possible the identification of each of the A, T, C and G haploid genotypes. Likewise, since rows 2-11 each predict a unique pattern of incorporation of dNMP-R label in the A/T/G/C Mixes in FIG. 19, the amounts of label incorporation predicted in this figure make possible the identification of each of the A/A, T/T, C/C and G/G homozygous diploid genotypes, as well as each of the A/T, A/C, A/G, T/C, T/G and C/G heterozygous diploid genotypes.

It is clear from the description above that many reaction combinations may be designed based on the present invention using solely non-chain terminating dNTPs or dNTP-Rs as Bridging Nucleotides, and non-chain-terminating dNTP-Rs as Reporting Nucleotides. Thus while the present invention is specifically described with reference to the examples given in FIGS. 1, 6 and 7, it should be understood that these examples are for illustration only and should not be taken as limitation on the invention. It is contemplated that many changes and modifications may be made by one of ordinary skill in the art without departing from the spirit and the scope of the invention described.

For example, although FIGS. 6 and 7 describe the use of PCR reaction as an amplification step, followed by nucleic acid purification to separate the PCR primers from the amplified products, it should be understood that should Primers 1 and 2 be designed in such a way as to be distinguishable from Primer 3, it becomes unnecessary even to have the purification step. For instance, if Primers 1 and 2 are of a length that is substantially longer than Primer 3, a technique that is capable of distinguishing between the three different primers and their polymerase-extended products by size would be able to produce informative data regarding chain extension of Primer 3 even in the presence of Primers 1 and 2, without the purification step after DNA amplification to remove Primers 1 and 2. In general, if size detection by mass spectrometry is used to distinguish between the polymerase-extended products, a Primer 3 of less than 50 bases would be preferred, as most mass spectroscopic methods work well only with DNA fragments not much longer than 50 base pairs. If capillary electrophoresis is used as the separation method for analyzing the length of primer extension, a primer of less than 100 bases in length would be advantageous. Thus the optimal length of Primer 3 to be employed depends on the method of size detection used and may be determined by the end user. As a non-limiting example, extension primer in FIG. 1, or Primer 3 in FIG. 7, could be 15-55 bases in length.

It is thus clear that the extension primer used in FIG. 1 and Primer 3 used in FIG. 7 may be designed along with the hybridization conditions to be employed. In FIGS. 6 and 7, it should be understood that the position of Sequence 2 may be any distance 5′ upstream, and Sequence 1 any distance 3′ downstream, of the SNP site as long as the SNP site and the base 5′ adjacent to the SNP are both amplified, and the amplified product has sufficient length for hybridization with Primer 3, and to enable the polymerase to catalyze the elongation of Primer 3. Preferably there should be a minimum of 100 bases between Sequence 2 and the SNP site.

The PCR reaction in FIG. 6 can be symmetrical, meaning that the two amplification primers are present at roughly equal molar concentrations, or it can be asymmetrical, in which one of the primers is added in excess.

For the chain extension reactions described in FIGS. 2-5 and 8-19, each extension reaction may be carried out in a single cycle of template-primer annealing and chain extension, or in multiple thermal cycles interspersed with thermal melting of the template-primer hybrid to increase sensitivity. Post-extension treatment by shrimp alkaline phosphatase or calf intestinal alkaline phosphatase will remove unincorporated dNTP-R after each thermal cycle. Such treatment may be needed in cases, for instance, where incorporation of fluorescence labeled dNMP-R is monitored by capillary electrophoresis, since left untreated the unincorporated fluorescent dNTP-R may overlap and interfere with the primer in the capillary electrophoretogram. Removal of the 5′ phosphoryl groups by phosphatase treatment alters the migration of the unincorporated fluorescent dNTP-R and thus may reduce interference. Such treatment may be performed just before the detection of extension products, and may not be needed for every thermal cycle.

From the foregoing description, it is clear that the present invention has multiple advantages:

The use of solely non-chain-terminating Bridging Nucleotides and Reporting Nucleotides, together with the uniform usage of the same R label group on different dNTP-R nucleotides in the present invention avoids the gross kinetic bias known to be displayed by DNA polymerases toward different kinds of chain-terminating nucleotides such as the dideoxy ddNTPs, and also minimizes any kinetic bias displayed by DNA polymerases toward unnatural nucleotides covalently bonded to different kinds of R groups including fluorescent dyes and non-fluorescent dyes.

Methods for genotyping target nucleotides based on the experimental differentiation between zero-base and single-base extensions are often difficult to implement with precision using mass spectrometry to measure the length of an extension primer because the addition of a single base to a multi-base primer results in only a modest percentile increase in the molecular weight of the extended primer. In contrast, in the present invention, as illustrated in FIGS. 2, 3, 8, 9, 12, 13, 16 and 17, genotyping by extension chain length is based on differentiation between zero-base extension and an extension of one, two or more bases, thus improving the ease and accuracy of detection of primer extensions by means of for example capillary electrophoresis or mass spectrometry.

Where label incorporation based on fluorescence or fluorescence polarization is employed to detect primer extension, the use of only one kind of fluorophore-labeled R group in the form of dNTP-R in the present invention may be implemented more readily and accurately compared to the use of more than one kind of fluorophore-labeled dNTP-Rs as in some other detection methods, because there is no inaccuracy arising from an incomplete resolution between the emission spectra of different fluorophores. Furthermore, the absence of any need for spectral resolution between different fluorophores also renders unnecessary the use of expensive fluorescence polarization readers equipped with high spectral resolution, thereby significantly reducing the equipment cost with respect to fluorescence or fluorescence polarization readers.

FIGS. 2-5 describe embodiment of the present invention for identifying the nature of SNP nucleotides on template DNAs that have not been amplified by PCR, and FIGS. 8-19 describe embodiment of the present invention for identifying the nature of SNP nucleotides on template DNAs that have been amplified by PCR. Both FIGS. 2-5 and FIGS. 8-19 depend on examining the chemical nature of an extension primer in solution phase after its incubation in each of the four different polymerase reaction mixtures A-Mix, T-Mix, G-Mix and C-Mix, with respect to the length of the extension primer or the amount of “R”-labeled nucleotidyl residues incorporated into the extension primer from a dNTP-R Reporting Nucleotide or Bridging Nucleotide. Alternatively, the chemical nature of the extension primer may be examined in solid phase following its incubation in a polymerase mixture. In one embodiment of this solid phase approach, the 5′ end of the Extension Primer described in FIG. 1, or the 5′ end of Primer 3 described in FIG. 7 is immobilized on to a solid surface. Extension of the immobilized primer is initiated by its hybridization with unamplified or PCR-amplified template DNA containing a SNP site, and addition of a polymerase reaction mix containing appropriate Bridging Nucleotide dNTP or dNTP-R, DNA polymerase, with or without also a Reporting Nucleotide dNTP-R. In this instance, it would be more difficult to analyze and distinguish between different lengths of primer extension. Therefore the predictions of label incorporation described in FIGS. 4, 5, 10, 11, 14, 15, 18 and 19 would be more easily applied than the predictions of extension length described in FIGS. 2, 3, 8, 9, 12, 13, 16 and 17. It would be convenient, although not essential, to implement the conditions of Mix A, Mix T, Mix G and Mix C in separate incubations, in each instance adding the polymerase reaction mixture containing an SNP (or target nucleotide)-containing template DNA segment from a human subject, an animal, a plant or a microbial genome to an addressed well on for example a 96 or 384-well array bearing a pre-immobilized extension primer targeted to a specific SNP site, and carrying out the extension reaction in either a single thermal cycle or multiple thermal cycles of template-primer annealing and chain extension. After washing away free nucleotidyl materials following the extension reaction, the amount of “R” label incorporated into the pre-immobilized extension primer can be determined at once for an entire array of primers, e.g. by using a Bridging Nucleotide dNTP or dNTP-R with or without also a Reporting dNTP-R, where “R” represents a fluorophore, and measuring the amount of “R” incorporation be means of a fluorescence plate reader.

In another embodiment of the solid state approach, the extension primer is first incubated in solution phase with SNP-containing template DNA in one of the polymerase reaction mixtures A-Mix, T-Mix, G-Mix and C-Mix under the reaction conditions as described in FIG. 4, 5, 10, 11, 14, 15, 18 or 19. Subsequently, the extension primer is melted from the template DNA, and hybridized to an ‘extension primer-hybridizing’ single-stranded DNA that has been pre-immobilized on a solid surface and contains a sequence segment that is complementary to the extension primer. After washing away free nucleotidyl materials including free dNTP-R as well as unhybridized extension primer from the solid surface, the amount of “R” label derived from labeled dNTP-R that has been incorporated into the extension primer and thereby captured through hybridization to the ‘extension primer-hybridizing’ single stranded DNA pre-immobilized on the solid surface can be estimated, e.g. by means of a fluorescence plate reader in the case of a fluorescent “R” label. This will yield a measure of “R” label incorporation into the extension primer, thus providing a basis for identifying the SNP nucleotide on the SNP-containing template DNA as described for FIG. 4, 5, 10, 11, 14, 15, 18 or 19. This procedure will be especially facilitated if the segment of the extension primer captured through hybridization by the immobilized ‘extension primer-hybridizing’ single-stranded DNA either does not hybridize, or hybridizes only minimally, with the template DNA, so that the template DNA will not interfere substantially with the capture of extension primer by the immobilized ‘extension primer-hybridizing’ single-stranded DNA.

The present invention allows considerable variations in its range of application. While the present invention is specifically described with reference to the examples described in FIGS. 1, 6 and 7, it should be understood that these examples are for illustration only and should not be taken as limitation on the invention. Many changes and modifications may be made by one of ordinary skill in the art without departing from the spirit and the scope of the invention.

### REFERENCES CITED

The following U.S. Patent documents and publications are incorporated by reference herein.

### U.S. PATENT DOCUMENTS

U.S. Pat. No. 6,972,174 issued on Dec. 6, 2005 with Xue and Wong listed as inventors.
U.S. Pat. No. 6,110,709 issued on Aug. 29, 2000 with Ausubel, et al. listed as inventors.

### FOREIGN PATENT DOCUMENTS

International Patent Publication WO 9303312 published on Feb. 18, 1993, with Cleveland Range, Inc. listed as the applicant.
International Patent Publication WO 0020853 published on Apr. 13, 2000, with City of Hope listed as the applicant.

### NON-PATENT LITERATURE

Current Protocols in Molecular Biology Editors Frederick M. Ausubel Roger Brent Robert E. Kingston David D. Moore, Massachusetts General Hospital and Harvard Medical School, Boston, Mass., USA J. G. Seidman Kevin Struhl, Harvard Medical School, Boston, Mass., USA John A. Smith, University of Alabama, Birmingham, Ala., USA. (2000)

What is claimed is:

1. A method for identifying a target nucleotide in a nucleic acid molecule, the nucleic acid molecule having a 3′ sequence and a 5′ sequence, wherein the target nucleotide is located between the 3′ sequence and the 5′ sequence of the nucleic acid molecule, comprising the steps of:
(a) mixing an oligonucleotide extension primer with the nucleic acid molecule in a plurality of reaction containers, wherein the oligonucleotide extension primer comprises a 3′ hydroxy terminus residue complementary to the 3′ sequence in the nucleic acid molecule directly adjacent to the target nucleotide on the 3′ side;
(b) allowing the 3′ hydroxy terminus of the oligonucleotide extension primer to hybridize with the 3′ sequence in the nucleic acid molecule;
(c) providing a distinct chain-extending Bridging Nucleotide to each of the reaction containers, such that the distinct Bridging Nucleotide in each container is complementary to a different possible nucleotidyl residue at the target nucleotide site;
(d) conducting a template dependent extension of the oligonucleotide extension primer by adding a polymerase reaction mixture to each of the reaction containers to give an extended primer; and
(e) measuring the incorporation of nucleotides into the extended primer in each of the reaction containers in order to identify the target nucleotide.

2. The method of claim 1, wherein the identity of the target nucleotide is determined by detecting the extended primer size.

3. The method of claim 1, wherein the identity of the target nucleotide is determined by detecting the amount of Reporting Nucleotide residue incorporated into the extended primer.

4. The method of claim 1, further comprising the step of:
adding a chain-extending Reporting Nucleotide to each reaction container before conducting a template dependent extension of the oligonucleotide extension primer, wherein the Reporting Nucleotide is complementary to a 5′ adjacent nucleotide in the nucleic acid molecule that is directly adjacent to the target nucleotide on the 5′ side.

5. The method of claim 4, wherein the Reporting Nucleotide is the same as the Bridging Nucleotide in one of the plurality of reaction containers.

6. The method of claim 1, wherein the identity of the target nucleotide is determined by detecting the amount of Bridging Nucleotide residue incorporated into the extended primer.

7. The method of claim 4, wherein the identity of the target nucleotide is determined by detecting the total amounts of Reporting Nucleotide residue and Bridging Nucleotide residue incorporated into the extended primer.

8. The method of claim 1, wherein the target nucleotide is a single nucleotide polymorphism (“SNP”).

9. The method of claim 1, wherein the target nucleotide is a point mutation.

10. The method of claim 1, wherein the nucleic acid molecule comprises an isolated genomic deoxyribonucleic acid (“DNA”) molecule.

11. The method of claim 10, wherein the isolated genomic DNA molecule contains a haploidal target SNP or point mutation.

12. The method of claim 10, wherein the isolated genomic DNA molecule contains a diploidal target SNP or point mutation.

13. The method of claim 1, wherein the nucleic acid molecule comprises a polymerase chain reaction (“PCR”) amplified DNA molecule.

14. The method of claim 13, wherein the PCR amplified DNA molecule contains a haploidal target SNP or point mutation.

15. The method of claim 13, wherein the PCR amplified DNA molecule contains a diploidal target SNP or point mutation.

16. The method of claim 1, wherein the oligonucleotide extension primer has a length in the range of about 15 to 55 nucleic acid residues.

17. The method of claim 1, wherein the oligonucleotide extension primer comprises a 5′ end attached to a solid surface.

18. The method of claim 1, wherein the oligonucleotide extension primer is capable of hybridizing with an extension primer-hybridizing single stranded DNA sequence attached to a solid surface.

19. The method of claim 1, wherein the chain-extending Bridging Nucleotide comprises a deoxyribonucleoside triphosphate (“dNTP”) compound.

20. The method of claim 19, wherein the dNTP is a natural compound or derivative thereof.

21. The method of claim 19, wherein the dNTP comprises a 2′-deoxyribonucleoside 5′-triphosphate.

22. The method of claim 1, wherein the chain-extending Bridging Nucleotide comprises a deoxyribonucleoside triphosphate (“dNTP-R”) compound.

23. The method of claim 22, wherein the dNTP is a natural compound or derivative thereof.

24. The method of claim 22, wherein the dNTP comprises a 2′-deoxyribonucleoside 5′-triphosphate.

25. The method of claim 22, wherein the dNTP-R contains a fluorescence labeled reporter group.

26. The method of claim 4, wherein the chain-extending Reporting Nucleotide comprises a deoxyribonucleoside triphosphate (“dNTP-R”) compound.

27. The method of claim 26, wherein the dNTP is a natural compound or derivative thereof.

28. The method of claim 26, wherein the dNTP comprises a 2′-deoxyribonucleoside 5′-triphosphate.

29. The method of claim 26, wherein the dNTP-R contains a fluorescence labeled reporter group.

30. The method of claim 1, wherein the polymerase reaction mixture comprises a nucleic acid polymerase and a buffer.

31. The method of claim 1, wherein measurement of the incorporation of nucleotides into the extended primer comprises determining a molecular mass of the extended primer.

32. The method of claim 31, wherein determining the molecular mass of the extended primer comprises an electrophoretic mobility analysis.

33. The method of claim 31, wherein determining the molecular mass of the extended primer comprises mass spectrometry analysis.

34. The method of claim 1, wherein measurement of the incorporation of nucleotides into the extended primer comprises determining the amount of fluorescence labeled nucleotides in the extended primer.

35. The method of claim 34, wherein determining the amount of fluorescence labeled nucleotides incorporated into the extended primer comprises measurement of fluorescence.

36. The method of claim 34, wherein determining the amount of fluorescence labeled nucleotides incorporated in the extended primer comprises measurement of fluorescence polarization.

37. The method of claim 34, wherein detecting the incorporation of nucleotides into the extended primer comprises determining the amount of fluorescence labeled nucleotide that has been incorporated into the extended primer in solution.

38. The method of claim 34, wherein detecting the incorporation of nucleotides into the extended primer comprises determining the amount of fluorescence labeled nucleotide that has been incorporated into the extended primer the 5′ end of which is pre-immobilized on a solid surface.

39. The method of claim 34, wherein detecting the incorporation of nucleotides into the extended primer comprises determining the amount of fluorescence labeled nucleotide that has been incorporated into the extended primer in solution following the capture of the extended primer through hybridization to a single-stranded DNA segment that has been pre-immobilized to a solid surface and is complementary to part or all of the oligonucleotide extension primer sequence.

40. The method of claim 1, wherein identifying the target nucleotide in the nucleic acid molecule comprises comparison of the experimentally determined incorporation of nucleotides into the extended primer with a predicted incorporation of nucleotides into the extended primer in each of the different reaction containers.

41. The method of claim 1, wherein identifying the target nucleotide in the nucleic acid molecule comprises comparison of the experimentally determined amount of incorporation of a fluorescence or isotope labeled nucleotide into the extended primer with a predicted amount of incorporation of the fluorescence or isotope labeled nucleotide into the extended primer in each of the different reaction containers.


SEQUENCE LISTING (4 sequences)

SEQ ID NO: 1 - DNA, 11 bp, Artificial Sequence
  template DNA
ctagnttcyyy

SEQ ID NO: 2 - DNA, 11 bp, Artificial Sequence
  amplified template DNA
gcaangctyyy

SEQ ID NO: 3 - DNA, 10 bp, Artificial Sequence
  predicted primer extension
gtttcgannn

SEQ ID NO: 4 - DNA, 10 bp, Artificial Sequence
  predicted primer extension
gttgcgannn